SEQUENCE LISTING (121 sequences)

SEQ ID NO: 1 - protein, 36 aa, Leiurus quinquestriatus
  misc_feature: Chlorotoxin
MCMPCFTTDHQMARKCDDCCGGKGRGKCYGPQCLCR

SEQ ID NO: 2 - protein, 42 aa, Leiurus quinquestriatus
HHHHHHMCMPCFTTDHQMARKCDDCCGGKGRGKCYGPQCLCR

SEQ ID NO: 3 - protein, 37 aa, Leiurus quinquestriatus
YMCMPCFTTDHQMARKCDDCCGGKGRGKCYGPQCLCR

SEQ ID NO: 4 - protein, 39 aa, Leiurus quinquestriatus
YSYMCMPCFTTDHQMARKCDDCCGGKGRGKCYGPQCLCR

SEQ ID NO: 5 - protein, 36 aa, Artificial sequence
  Synthetic - chlorotoxin variant
MCMPCFTTDHQMARKCDDCCGGKGRGKCFGPQCLCR

SEQ ID NO: 6 - protein, 35 aa, Artificial sequence
  Synthetic - chlorotoxin variant
RCKPCFTTDPQMSKKCADCCGGKGKGKCYGPQCLC

SEQ ID NO: 7 - protein, 38 aa, Artificial sequence
  Synthetic - chlorotoxin variant
RCSPCFTTDQQMTKKCYDCCGGKGKGKCYGPQCICAPY

SEQ ID NO: 8 - protein, 7 aa, Leiurus quinquestriatus
  misc_feature: Derivative of Chlorotoxin  amino acid residues 23-29
KGRGKSY

SEQ ID NO: 9 - protein, 7 aa, Leiurus quinquestriatus
  misc_feature: Derivative of Chlorotoxin  amino acid residues 8-14
TDHQMAR

SEQ ID NO: 10 - protein, 9 aa, Artificial sequence
  Synthetic - chlorotoxin alpha peptide
TDHQMARKS

SEQ ID NO: 11 - protein, 9 aa, Artificial sequence
  Synthetic - variant of chlorotoxin alpha peptide
TAHAMARKS

SEQ ID NO: 12 - protein, 36 aa, Artificial sequence
  Synthetic - variant peptide of chlorotoxin
MCMPCFTTAHAMARKCDDCCGGKGRCKCYGPQCLCR

SEQ ID NO: 13 - protein, 9 aa, Artificial
  Synthetic - motif for chlorotoxin derivatives
TTXXXMXXK

SEQ ID NO: 14 - protein, 9 aa, Leiurus quinquestriatus
TTDHQMARK

SEQ ID NO: 15 - protein, 35 aa, Mesobuthus tamulus; identical to SEQ ID NO: 6

SEQ ID NO: 16 - protein, 34 aa, Artificial sequence
  Small Toxin consensus sequence
CXPCFTTDXQMAKKCXDCCGGKGKGKCYGPQCLC

SEQ ID NO: 17 - protein, 38 aa, Leiurus quinquestriatus; identical to SEQ ID NO: 7

SEQ ID NO: 18 - protein, 34 aa, Artificial sequence
  Synthetic - Probable Toxin LQH 8/6 consensus sequence
CXPCFTTDXQMXKKCXDCCGGKGKGKCYGPQCIC

SEQ ID NO: 19 - protein, 61 aa, Mesobuthus martensii
  MISC_FEATURE (1)..(61): Xaa can be any amino acid
MKFLYGIVFIALFLTVMFATQTDGCGPCFTTDANMARKCRECCGGIGXXKCFGPQCLCNRI

SEQ ID NO: 20 - protein, 34 aa, Artificial sequence
  Synthetic - Chinese Scorpion consensus sequence
CXPCFTTDXNMARKCXDCCGGXGXXKCFGPQCLC

SEQ ID NO: 21 - protein, 59 aa, Mesobuthus martensii
MKFLYGIVFIALFLTVMFATQTDGCGPCFTTDANMARKCRECCGGIGKCFGPQCLCNRI

SEQ ID NO: 22 - protein, 32 aa, Artificial sequence
  Chinese Scorpion consensus sequence
CXPCFTTDXNMARKCXDCCGGXGKXXXXXXXC

SEQ ID NO: 23 - protein, 37 aa, Mesobuthus eupeus
  MISC_FEATURE (1)..(37): Xaa can be any amino acid
MCMPCFTTDPNMANKCRDCCGGXGKXKCFGPQCLCNR

SEQ ID NO: 24 - protein, 35 aa, Artificial sequence
  Synthetic - Insect toxin I5 consensus sequence
MCMPCFTTDXNMAXKCXDCCGGXGKXKCFGPQCLC

SEQ ID NO: 25 - protein, 35 aa, Mesobuthus eupeus
MCMPCFTTDPNMANKCRDCCGGGKKCFGPQCLCNR

SEQ ID NO: 26 - protein, 33 aa, Artificial sequence
  Synthetic - Insect toxin I5 consensus sequence
MCMPCFTTDXNMAXKCXDCCGGXXKXXXXXXXC

SEQ ID NO: 27 - protein, 38 aa, Mesobuthus eupeus
  MISC_FEATURE (1)..(38): Xaa can be any amino acid
MCMPCFTTRPDMAQQCRACCKGXXRGKCFGPQCLCGYD

SEQ ID NO: 28 - protein, 35 aa, Artificial sequence
  Synthetic - Insectotoxin I1 consensus sequence
MCMPCFTTXXXMAXXCXXCCXGXXRGKCFGPQCLC

SEQ ID NO: 29 - protein, 36 aa, Mesobuthus eupeus
MCMPCFTTRPDMAQQCRACCKGRGKCFGPQCLCGYD

SEQ ID NO: 30 - protein, 33 aa, Artificial sequence
  Synthetic - Insectotoxin I1 consensus sequence
MCMPCFTTXXXMAXXCXXCCXGKGKXXXXXXXC

SEQ ID NO: 31 - protein, 37 aa, Mesobuthus eupeus
  MISC_FEATURE (1)..(37): Xaa can be any amino acid
MCMPCFTTDPNMAKKCRDCCGGNGXXKCFGPQCLCNR

SEQ ID NO: 32 - protein, 35 aa, Artificial sequence
  Synthetic - Insectotoxin 15A consensus sequence
MCMPCFTTDXNMAKKCXDCCGGXGXXKCFGPQCLC

SEQ ID NO: 33 - protein, 35 aa, Mesobuthus eupeus
MCMPCFTTDPNMAKKCRDCCGGNGKCFGPQCLCNR

SEQ ID NO: 34 - protein, 33 aa, Artificial sequence
  Synthetic - Insectotoxin 15A consensus sequence
MCMPCFTTDXNMAKKCXDCCGGXGKXXXXXXXC

SEQ ID NO: 35 - protein, 37 aa, Androctonus mauretanicus
  MISC_FEATURE (1)..(37): Xaa can be any amino acid
CGPCFTTDPYTESKCATCCGGXXRGKCVGPQCLCNRI

SEQ ID NO: 36 - protein, 34 aa, Artificial sequence
  Neurotoxin P2 consensus sequence
CXPCFTTDXXXXXKCXXCCGGXXRGKCXGPQCLC

SEQ ID NO: 37 - protein, 35 aa, Androctonus mauretanicus
CGPCFTTDPYTESKCATCCGGRGKCVGPQCLCNRI

SEQ ID NO: 38 - protein, 32 aa, Artificial sequence
  Synthetic - Neurotoxin P2 consensus sequence
CXPCFTTDXXXXXKCXXCCGGKGKXXXXXXXC

SEQ ID NO: 39 - protein, 37 aa, Artificial sequence
  Synthetic - Toxin consensus sequence
MCMPCFTTDPNMAKKCRDCCGGKGXXKCFGPQCLCNR

SEQ ID NO: 40 - protein, 35 aa, Artificial sequence
  Synthetic - Toxin consensus sequence
RCXPCFTTDXQMSKKCXDCCGGKGKGKCYGPQCLC

SEQ ID NO: 41 - protein, 35 aa, Artificial sequence
  Synthetic -Toxin consensus sequence
MCMPCFTTDPNMARKCRDCCGGRGKCFGPQCLCNR

SEQ ID NO: 42 - protein, 10 aa, Artificial sequence
  Synthetic - Pep8-Ctlx
CGGKGRGKCY

SEQ ID NO: 43 - protein, 10 aa, Artificial sequence
  Synthetic - Pep8-SCX1_BUTSI
CGGKGKGKCY

SEQ ID NO: 44 - protein, 10 aa, Artificial sequence
  Synthetic - Pep8-AF079059_2
CGGIGKCFGP

SEQ ID NO: 45 - protein, 12 aa, Artificial sequence
  Synthetic - Chlorotoxin Peptide 8 consensus sequence
CGGXGRGKCFGP

SEQ ID NO: 46 - protein, 6 aa, Artificial sequence
  Synthetic - Chlorotoxin Peptide 8 consensus sequence
CGGXGK

SEQ ID NO: 47 - protein, 10 aa, Artificial sequence
  Synthetic - Pep8-NJ0361 sequence
CGGGKKCFGP

SEQ ID NO: 48 - protein, 12 aa, Artificial sequence
  Synthetic - Chlorotoxin Peptide 8 consensus sequence
CGGKGKGKCFGP

SEQ ID NO: 49 - protein, 6 aa, Artificial sequence
  Synthetic - Chlorotoxin Peptide 8 consensus sequence
CGGXXK

SEQ ID NO: 50 - protein, 10 aa, Artificial sequence
  Synthetic - Pep8-SCX1_BUTEU sequence
CKGRGKCFGP

SEQ ID NO: 51 - protein, 12 aa, Artificial sequence
  Synthetic - chlorotoxin peptide 8 consensus sequence
CGXKGRGKCFGP

SEQ ID NO: 52 - protein, 6 aa, Artificial sequence
  Synthetic - chlorotoxin peptide 8 consensus sequence
CXGKGK

SEQ ID NO: 53 - protein, 10 aa, Artificial sequence
  Synthetic - Pep8-SCX5_BUTEU sequence
CGGNGKCFGP

SEQ ID NO: 54 - protein, 12 aa, Artificial sequence
  Synthetic - chlorotoxin peptide 8 consensus sequence
CGGXGRGKCFGP

SEQ ID NO: 55 - protein, 6 aa, Artificial sequence
  Synthetic - chlorotoxin peptide 8 consensus sequence
CGGXGK

SEQ ID NO: 56 - protein, 10 aa, Artificial sequence
  Synthetic - Pep8-SCXP_ANDMA sequence
CGGRGKCVGP

SEQ ID NO: 57 - protein, 12 aa, Artificial sequence
  Synthetic - chlorotoxin peptide 8 consensus sequence
CGGKGRGKCXGP

SEQ ID NO: 58 - protein, 6 aa, Artificial sequence
  Synthetic - chlorotoxin peptide 8 consensus sequence
CGGKGK

SEQ ID NO: 59 - protein, 12 aa, Artificial sequence
  Synthetic - chlorotoxin peptide 8 consensus sequence
CGGXXRGKCFGP

SEQ ID NO: 60 - protein, 10 aa, Artificial sequence
  Synthetic - chlorotoxin peptide 8 consensus sequence
CGGKGKCFGP

SEQ ID NO: 61 - protein, 10 aa, Artificial sequence
  Synthetic - chlorotoxin peptide 21 sequence
TTDHQMARKC

SEQ ID NO: 62 - protein, 10 aa, Artificial sequence
  Synthetic - Pep21-SCX1-BUTSI sequence
TTDPQMSKKC

SEQ ID NO: 63 - protein, 10 aa, Artificial sequence
  Synthetic - chlorotoxin peptide 21 consensus sequence
TTDXQMAKKC

SEQ ID NO: 64 - protein, 10 aa, Artificial sequence
  Synthetic - Pep21-SCX8_LEIQH sequence
TTDQQMTKKC

SEQ ID NO: 65 - protein, 10 aa, Artificial sequence
  Synthetic - chlorotoxin peptide 21 consensus sequence
TTDXQMXKKC

SEQ ID NO: 66 - protein, 10 aa, Artificial sequence
  Synthetic - Pep21-AF079059_2 sequence
TTDANMARKC

SEQ ID NO: 67 - protein, 10 aa, Artificial sequence
  Synthetic - chlorotoxin peptide 21 consensus sequence
TTDXNMARKC

SEQ ID NO: 68 - protein, 10 aa, Artificial sequence
  Synthetic - Pep21-JN0361 sequence
TTDPNMANKC

SEQ ID NO: 69 - protein, 10 aa, Artificial sequence
  Synthetic - chlorotoxin peptide 21 consensus sequence
TTDXNMAXKC

SEQ ID NO: 70 - protein, 10 aa, Artificial sequence
  Synthetic - Pep21-SCX1_BUTEU sequence
TTRPDMAQQC

SEQ ID NO: 71 - protein, 10 aa, Artificial sequence
  Synthetic - chlorotoxin peptide 21 consensus sequence
TTXXXMAXXC

SEQ ID NO: 72 - protein, 10 aa, Artificial sequence
  Synthetic - Pep21-SCX5_BUTEU sequence
TTDPNMAKKC

SEQ ID NO: 73 - protein, 10 aa, Artificial sequence
  Synthetic - chlorotoxin peptide 21 consensus sequence
TTDXNMAKKC

SEQ ID NO: 74 - protein, 10 aa, Artificial sequence
  Synthetic - Pep21-SCXP_ANDMA sequence
TTDPYTESKC

SEQ ID NO: 75 - protein, 10 aa, Artificial sequence
  Synthetic - chlorotoxin peptide 21 consensus sequence
TTDXXXXXKC

SEQ ID NO: 76 - protein, 10 aa, Artificial sequence
  Synthetic - chlorotoxin peptide 21 consensus sequence
TTDPNMAKKC

SEQ ID NO: 77 - protein, 7 aa, Artificial sequence
  Synthetic - chlorotoxin derivative STP-1
TDPQMSR

SEQ ID NO: 78 - protein, 10 aa, Artificial sequence
  Synthetic - peptide 8 sequences
GGKGRGKSYG

SEQ ID NO: 79 - protein, 9 aa, Artificial sequence
  Synthetic - peptide 8a sequence
GKGRGKSYG

SEQ ID NO: 80 - protein, 8 aa, Artificial sequence
  Synthetic - peptide 8b sequence
KGRGKSYG

SEQ ID NO: 81 - protein, 7 aa, Artificial sequence
  Synthetic - peptide 8c sequence
GRGKSYG

SEQ ID NO: 82 - protein, 10 aa, Artificial sequence
  Synthetic - peptide 21 sequence
TTDHQMARKS

SEQ ID NO: 83 - protein, 8 aa, Artificial sequence
  Synthetic - peptide 21b sequence
DHQMARKS

SEQ ID NO: 84 - protein, 7 aa, Artificial sequence
  Synthetic - peptide 21c sequence
HQMARKS

SEQ ID NO: 85 - protein, 6 aa, Artificial sequence
  Synthetic - peptide 21d sequence
QMARKS

SEQ ID NO: 86 - protein, 9 aa, Artificial sequence
  Synthetic - peptide 21a-A1 sequence
ADHQMARKS

SEQ ID NO: 87 - protein, 9 aa, Artificial sequence
  Synthetic - peptide 21a-A2 sequence
TAHQMARKS

SEQ ID NO: 88 - protein, 9 aa, Artificial sequence
  Synthetic - peptide 21a-A3 sequence
TDAQMARKS

SEQ ID NO: 89 - protein, 9 aa, Artificial sequence
  Synthetic - peptide 21a-A4 sequence
TDHAMARKS

SEQ ID NO: 90 - protein, 9 aa, Artificial sequence
  Synthetic - peptide 21a-A5 sequence
TDHQAARKS

SEQ ID NO: 91 - protein, 9 aa, Artificial sequence
  Synthetic - peptide 21a-A7 sequence
TDHQMAAKS

SEQ ID NO: 92 - protein, 9 aa, Artificial sequence
  Synthetic - peptide 21a-A8 sequence
TDHQMARAS

SEQ ID NO: 93 - protein, 9 aa, Artificial sequence
  Synthetic - peptide 21a-A9 sequence
TDHQMARKA

SEQ ID NO: 94 - protein, 9 aa, Mesobuthus tamulus sindicus
  misc_feature (1)..(9): GenBank Accession No. P15229, small toxin
TTDQQMSKK

SEQ ID NO: 95 - protein, 9 aa, Leiurus quinquestriatus hebraeu
  misc_feature (1)..(9): GenBank Accession No. P55966, probable toxin
TTDPQMSKK

SEQ ID NO: 96 - protein, 10 aa, Artificial Sequence
  Synthetic fragment derived from chlorotoxin (Leiurus quinquestiratus)
KSYGPQSLSR

SEQ ID NO: 97 - protein, 10 aa, Artificial Sequence
  Synthetic fragment derived from chlorotoxin (Leiurus quinquestiratus)
GKSYGPQSLS

SEQ ID NO: 98 - protein, 10 aa, Artificial Sequence
  Synthetic fragment derived from chlorotoxin (Leiurus quinquestiratus)
RGKSYGPQSL

SEQ ID NO: 99 - protein, 10 aa, Artificial Sequence
  Synthetic fragment derived from chlorotoxin (Leiurus quinquestiratus)
GRGKSYGPQS

SEQ ID NO: 100 - protein, 10 aa, Artificial Sequence
  Synthetic fragment derived from chlorotoxin (Leiurus quinquestiratus)
KGRGKSYGPQ

SEQ ID NO: 101 - protein, 10 aa, Artificial Sequence
  Synthetic fragment derived from chlorotoxin (Leiurus quinquestiratus)
GKGRGKSYGP

SEQ ID NO: 102 - protein, 10 aa, Artificial Sequence
  Synthetic fragment derived from chlorotoxin (Leiurus quinquestiratus)
GGKGRGKSYG

SEQ ID NO: 103 - protein, 10 aa, Artificial Sequence
  Synthetic fragment derived from chlorotoxin (Leiurus quinquestiratus)
SGGKGRGKSY

SEQ ID NO: 104 - protein, 10 aa, Artificial Sequence
  Synthetic fragment derived from chlorotoxin (Leiurus quinquestiratus)
SSGGKGRGKS

SEQ ID NO: 105 - protein, 10 aa, Artificial Sequence
  Synthetic fragment derived from chlorotoxin (Leiurus quinquestiratus)
DSSGGKGRGK

SEQ ID NO: 106 - protein, 10 aa, Artificial Sequence
  Synthetic fragment derived from chlorotoxin (Leiurus quinquestiratus)
DDSSGGKGRG

SEQ ID NO: 107 - protein, 10 aa, Artificial Sequence
  Synthetic fragment derived from chlorotoxin (Leiurus quinquestiratus)
SDDSSGGKGR

SEQ ID NO: 108 - protein, 10 aa, Artificial Sequence
  Synthetic fragment derived from chlorotoxin (Leiurus quinquestiratus)
KSDDSSGGKG

SEQ ID NO: 109 - protein, 10 aa, Artificial Sequence
  Synthetic fragment derived from chlorotoxin (Leiurus quinquestiratus)
RKSDDSSGGK

SEQ ID NO: 110 - protein, 10 aa, Artificial Sequence
  Synthetic fragment derived from chlorotoxin (Leiurus quinquestiratus)
ARKSDDSSGG

SEQ ID NO: 111 - protein, 10 aa, Artificial Sequence
  Synthetic fragment derived from chlorotoxin (Leiurus quinquestiratus)
MARKSDDSSG

SEQ ID NO: 112 - protein, 10 aa, Artificial Sequence
  Synthetic fragment derived from chlorotoxin (Leiurus quinquestiratus)
QMARKSDDSS

SEQ ID NO: 113 - protein, 10 aa, Artificial Sequence
  Synthetic fragment derived from chlorotoxin (Leiurus quinquestiratus)
HQMARKSDDS

SEQ ID NO: 114 - protein, 10 aa, Artificial Sequence
  Synthetic fragment derived from chlorotoxin (Leiurus quinquestiratus)
DHQMARKSDD

SEQ ID NO: 115 - protein, 10 aa, Artificial Sequence
  Synthetic fragment derived from chlorotoxin (Leiurus quinquestiratus)
TDHQMARKSD

SEQ ID NO: 116 - protein, 10 aa, Artificial Sequence
  Synthetic fragment derived from chlorotoxin (Leiurus quinquestiratus)
FTTDHQMARK

SEQ ID NO: 117 - protein, 10 aa, Artificial Sequence
  Synthetic fragment derived from chlorotoxin (Leiurus quinquestiratus)
SFTTDHQMAR

SEQ ID NO: 118 - protein, 10 aa, Artificial Sequence
  Synthetic fragment derived from chlorotoxin (Leiurus quinquestiratus)
PSFTTDHQMA

SEQ ID NO: 119 - protein, 10 aa, Artificial Sequence
  Synthetic fragment derived from chlorotoxin (Leiurus quinquestiratus)
MPSFTTDHQM

SEQ ID NO: 120 - protein, 10 aa, Artificial Sequence
  Synthetic fragment derived from chlorotoxin (Leiurus quinquestiratus)
SMPSFTTDHQ

SEQ ID NO: 121 - protein, 10 aa, Artificial Sequence
  Synthetic fragment derived from chlorotoxin (Leiurus quinquestiratus)
MSMPSFTTDH

(19) United States
(12) Patent Application Publication
(10) Pub. No.: US 2011/0091380 A1
(43) Pub. Date: Apr. 21, 2011

(54) CHLOROTOXINS AS DRUG CARRIERS

(75) Inventors: Douglas Jacoby, Wellesley, MA (US); Abdellah Sentissi, Dover, MA (US); E. Michael Egan, Brookline, MA (US)

(73) Assignee: TRANSMOLECULAR, INC., Cambridge, MA (US)

(21) Appl. No.: 12/672,069

(22) PCT Filed: Aug. 7, 2008
(86) PCT No.: PCT/US08/72524

§ 371 Date: Feb. 3, 2010

Related U.S. Application Data
(60) Provisional application No. 60/954,409, filed on Aug. 7, 2007.

(51) Int. Cl. A61K 51/08; C07K 14/435; C12N 9/96; A61K 38/17; A61P 35/00; A61K 38/43; A61K 31/7105; A61K 39/385

(52) U.S. Cl. 424/1.69; 530/300; 435/188; 530/324; 530/322; 514/19.3; 424/94.3; 514/44 A; 424/193.1

(57) ABSTRACT

The present invention relates to the use of a toxin moiety (e.g., a chlorotoxin moiety) as a carrier for therapeutic agents, e.g., therapeutic agents that require intracellular uptake to exert their effects. For example, in some embodiments, the present invention provides conjugates comprising a toxin (e.g., a chlorotoxin) moiety and an anti-cancer moiety and methods for using such conjugates to increase cellular uptake and/or increase specificity for cancer cells of the anti-cancer drug. In some embodiments, the present invention provides conjugates comprising a toxin moiety (e.g., a chlorotoxin moiety) and a nucleic acid agent. Also provided are methods of treatment involving administration of such conjugates, and pharmaceutical compositions and kits useful for carrying out such methods of treatment.

### RELATED APPLICATIONS

This application claims priority to and claims benefit of U.S. Provisional Application No. 60/954,409 filed Aug. 7, 2007, the entire contents of which are herein incorporated by reference.

### BACKGROUND OF THE INVENTION

The clinical use of chemotherapeutic agents against malignant tumors is successful in many cases but also has several limitations (B. A. Chabner and T. G. Roberts, Nature Rev. Cancer, 2005, 5: 65-72). In particular, anti-cancer drugs often do not affect tumor cells selectively over healthy cells, which leads to high toxicity and side effects (M. V. Blagosklonny, Trends Pharmacol. Sci., 2005, 26: 77-81). Tissues with high cellular division rates (e.g., bone marrow, intestinal mucosa, and the hair follicle cells) are particularly affected. The lack of selectivity and resulting adverse systemic toxicity limit the dose of drug that can be administered to a patient, and therefore the therapeutic potential of certain anti-cancer drugs.

Lack of selectivity is only one, albeit major, obstacle hindering the optimization of tumor drug effectiveness. The efficiency of chemotherapeutic drugs may also be seriously limited by the presence or development of cellular drug resistance (M. Pomeroy and M. Moriarty, Cytotechnology, 1993, 12: 385-391; G. Giaccone and H. M. Pinedo, The Oncologist, 1996, 1: 82-87; M. M. Gottesman, Ann. Rev. Medicine, 2002, 53: 615-627; G. D. Kruth, Oncogene, 2003, 22: 7262-7264). Resistance to a cytostatic/cytotoxic agent can operate by different mechanisms including reduced intracellular accumulation due to decrease or loss of plasma membrane carriers that results in certain anti-cancer drugs being prevented from entering cells and/or increase in the level of energy-dependent pumps such as p-glycoprotein resulting in extrusion of the drug from the tumor cell, premature inactivation of the drug leading to insufficient concentration at the target site, impaired activation of the drug due to decrease in or loss of specific enzymatic activities, formation of inactivating antibodies, and appearance of DNA repair mechanisms.

Another limitation of certain chemotherapeutics is their intrinsic low solubility in water. The membrane permeability and efficacy of such drugs increases with increasing hydrophobicity. In addition, parenteral administration of these hydrophobic agents is associated with some problems. Thus, intravenous administration of aggregates formed by undissolved drug in aqueous media can cause embolization of blood capillaries before the drug penetrates a tumor. Additionally, the low solubility of hydrophobic drugs in combination with excretion and metabolic degradation hinders the maintenance of therapeutically significant systemic concentrations.

The challenges that face chemotherapeutic agents can also be true of other therapeutic agents. In particular, effective delivery to cells remains problematic for many different therapeutic entities.

Although drug delivery systems have been developed with the goal of optimizing drug effectiveness, many of these systems (e.g., micelles, liposomes, microparticles, antibodies and drug-polymer conjugates) suffer from limitations including instability in the plasma, susceptibility to oxidation or other degradation mechanisms, technical problems with their production, rapid scavenging by reticuloendothelial cells, absence of or low selectivity for cancer cells, and limited cellular internalization. Therefore, there is still a need in the art for improved drug-delivery approaches to overcome the above-mentioned problems and substantially enhance drug delivery. Particularly desirable is the development of drug carriers or vehicles that can selectively deliver the drug into cells.

### SUMMARY OF THE INVENTION

The present invention is directed to new systems and strategies for improved delivery and administration of therapeutic agents (e.g., anti-cancer agents). In particular, the present invention encompasses the recognition that toxin moieties such as chlorotoxin (1) exhibit high specificity for cancer cells, (2) undergo efficient cellular internalization, and (3) remain stable in cells for a period of time. Accordingly, the present invention relates to the use of toxin moieties as carriers for therapeutic agents (e.g., chemotherapeutics, nucleic acid agents, etc). The present invention provides methods and compositions for the administration and delivery of drugs at their sites of action, for example tumor sites. The present invention provides systems for delivering therapeutic agents into cells. In certain embodiments, the present invention provides conjugates that comprise a toxin moiety (e.g., a chlorotoxin or a related agent) associated with a therapeutic agent (e.g., an anti-cancer agent).

Administration of an inventive conjugate to a patient may increase specificity for target cells (particularly for tumor cells), increase cellular internalization by cells, decrease cellular degradation by cells, increase accumulation at the target site, overcome drug resistance, increase biological activity of the drug, and/or prevent, limit or eliminate undesirable side effects and toxicity as compared with administration of the therapeutic agent alone (i.e., not as part of an inventive conjugate).

These and other objects, advantages, and features of the present invention will become apparent to those of ordinary skill in the art having read the following detailed description of the preferred embodiments.

### BRIEF DESCRIPTION OF THE DRAWING

FIG. 1 depicts a set of two fluorescence microscopy images demonstrating the rapid uptake and long-term intracellular localization of TM-601 within tumor cells. (A) shows the perinuclear localization of TM-601 (green) in non-fixed live cells. Nuclei appear in blue. (B) shows cells after removing the TM-601 from the media and culturing for an additional 6 days at 37° C.

### Definitions

Throughout the specification, several terms are employed that are defined in the following paragraphs.

The terms “individual” and “subject” are used herein interchangeably. They refer to a human or another mammal (e.g., mouse, rat, rabbit, dog, cat, cattle, swine, sheep, horse or primate) that can be afflicted with or is susceptible to a disease or disorder (e.g., cancer) but may or may not have the disease or disorder. In many embodiments, the subject is a human being. The terms “individual” and “subject” do not denote a particular age, and thus encompass adults, children, and newborns.

As used herein, the term “cancer patient” can refer to an individual suffering from or susceptible to cancer. Cancer patients may or may not have been diagnosed with cancer. The term also include individuals that have previously undergone therapy for cancer.

The term “treatment” is used herein to characterize a method or process that is aimed at (1) delaying the onset of a disease or condition; (2) slowing down or stopping the progression, aggravation, or deterioration of the symptoms of the disease or condition; (3) bringing about ameliorations of the degree and/or incidence of one or more symptoms of the disease or condition; (4) curing the disease or condition. A treatment may be administered prior to the onset of the disease, for a prophylactic action. Alternatively or additionally, treatment may be administered after initiation of the disease or condition, for a therapeutic action.

A “pharmaceutical composition” is defined herein as comprising an effective amount of at least one agent of the invention (i.e., a toxin conjugate), and at least one pharmaceutically acceptable carrier.

As used herein, the term “effective amount” refers to any amount of a compound, agent or composition that is sufficient to fulfill its intended purpose(s), e.g., a desired biological or medicinal response in a tissue, system or subject. For example, in certain embodiments of the present invention, the purpose(s) may be: to specifically deliver a drug to a target tissue, to deliver a drug inside a cell (e.g., a cancer cell), to treat a disease or disorder (e.g., cancer), etc.

As used herein, the term “physiologically tolerable salt” refers to any acid addition or base addition salt that retains the biological activity and properties of the corresponding free base or free acid, respectively, and that is not biologically or otherwise undesirable. Acid addition salts are formed with inorganic acids (e.g., hydrochloric, hydrobromic, sulfuric, nitric, phosphoric acids, and the like); and organic acids (e.g., acetic, propionic, pyruvic, maleic, malonic, succinic, fumaric, tartaric, citric, benzoic, mandelic, methanesulfonic, ethanesulfonic, p-toluenesulfonic, salicylic acids, and the like. Base addition salts can be formed with inorganic bases (e.g., sodium, potassium, lithium, ammonium, calcium, magnesium, zinc, aluminum salts, and the like) and organic bases (e.g., salts of primary, secondary and tertiary amines, substituted amines including naturally occurring substituted amines, cyclic amines, and basic ion exchange resins, such as isopropylamine, trimethylamine, diethylamine, triethylamine, tripropylamine, ethanolamine, 2-dimethyl-aminoethanol, 2-diethylaminoethanol, trimethamine, dicyclohexyl-amine, lysine, arginine, histidine, caffeine, procaine, hydrabanine, choline, betaine, ethylene-diamine, glycosamine, methylglucamine, theobromine, purines, piperazine, N-ethylpiperidine, polyamine resins, and the like).

As used herein, the term “pharmaceutically acceptable carrier” refers to a carrier medium which does not interfere with the effectiveness of the biological activity of the active ingredient(s) and which is not excessively toxic to the host at the concentration at which it is administered. The term includes solvents, dispersion media, coatings, antibacterial and antifungal agents, isotonic agents, absorption delaying agents, and the like. The use of such media and agents for pharmaceutically active substances is well known in the art (see for example, “Remington's Pharmaceutical Sciences”, E. W. Martin, 18^{th} Ed., 1990, Mack Publishing Co.: Easton, Pa., which is incorporated herein by reference in its entirety).

As used herein, the term “cancer cell” refers to a cell that undergoes unregulated cell growth. In some embodiments, a cancer cell is a cell in a mammal (e.g., a human being) in vivo which undergoes undesired and unregulated cell growth or abnormal persistence of abnormal invasion of tissues. In some embodiments, a cancer cell is a cell in vitro that is permanently immortalized (e.g., as a cell line established cell culture that will proliferate indefinitely and in an unregulated manner, if given appropriate fresh medium and space).

As used herein, the term “cancer” refers to or describes the physiological condition in mammals that is typically characterized by unregulated cell growth. Examples of cancers include, but are not limited to carcinoma, lymphoma, blastoma, sarcoma, and leukemia. More particularly, examples of such cancers include lung cancer, bone cancer, liver cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the sexual and reproductive organs, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the bladder, cancer of the kidney, renal cell carcinoma, carcinoma of the renal pelvis, neoplasms of the central nervous system (CNS), neuroectodermal cancer, spinal axis tumors, glioma, meningioma, and pituitary adenoma.

The terms “therapeutic agent” and “drug” are used herein interchangeably. They refer to a substance, molecule, compound, agent, factor or composition effective in the treatment of a disease or clinical condition.

The terms “chemotherapeutics” and “anti-cancer agents or drugs” are used herein interchangeably. They refer to those medications that are used to treat cancer or cancerous conditions. Anti-cancer drugs are conventionally classified in one of the following group: radioisotopes (e.g., Iodine-131, Lutetium-177, Rhenium-188, Yttrium-90), toxins (e.g., diphtheria, pseudomonas, ricin, gelonin), enzymes, enzymes to activate prodrugs, radio-sensitizing drugs, interfering RNAs, superantigens, anti-angiogenic agents, alkylating agents, purine antagonists, pyrimidine antagonists, plant alkaloids, intercalating antibiotics, aromatase inhibitors, anti-metabolites, mitotic inhibitors, growth factor inhibitors, cell cycle inhibitors, enzymes, topoisomerase inhibitors, biological response modifiers, anti-hormones and anti-androgens. Examples of such anti-cancer agents include, but are not limited to, BCNU, cisplatin, gemcitabine, hydroxyurea, paclitaxel, temozolomide, topotecan, fluorouracil, vincristine, vinblastine, procarbazine, decarbazine, altretamine, methotrexate, mercaptopurine, thioguanine, fludarabine phosphate, cladribine, pentostatin, cytarabine, azacitidine, etoposide, teniposide, irinotecan, docetaxel, doxorubicin, daunorubicin, dactinomycin, idarubicin, plicamycin, mitomycin, bleomysin, tamoxifen, flutamide, leuprolide, goserelin, aminogluthimide, anastrozole, amsacrine, asparaginase, mitoxantrone, mitotane and amifostine.

The term “prodrug” refers to a compound that, after in vivo administration, is metabolized or otherwise converted to the biologically, pharmaceutically or therapeutically active form of the compound. A prodrug may be designed to alter the metabolic stability or the transport characteristics of a compound, to mask side effects or toxicity, to improve the flavor of a compound and/or to alter other characteristics or properties of a compound. By virtue of knowledge of pharmacodynamic processes and drug metabolisms in vivo, once a pharmaceutically active compound is identified, those of skill in the pharmaceutical art generally can design prodrugs of the compound (Nogrady, “Medicinal Chemistry A Biochemical Approach”, 1985, Oxford University Press: N.Y., pages 388-392). Procedures for the selection and preparation of suitable prodrugs are also known in the art. In the context of the present invention, a prodrug is preferably a compound that, after in vivo administration, whose conversion to its active form involves enzymatic catalysis.

The terms “protein”, “polypeptide”, and “peptide” are used herein interchangeably, and refer to amino acid sequences of a variety of lengths, either in their neutral (uncharged) forms or as salts, and either unmodified or modified by glycosylation, side chain oxidation, or phosphorylation. In certain embodiments, the amino acid sequence is the full-length native protein. In other embodiments, the amino acid sequence is a smaller fragment of the full-length protein. In still other embodiments, the amino acid sequence is modified by additional substituents attached to the amino acid side chains, such as glycosyl units, lipids, or inorganic ions such as phosphates, as well as modifications relating to chemical conversion of the chains, such as oxidation of sulfhydryl groups. Thus, the term “protein” (or its equivalent terms) is intended to include the amino acid sequence of the full-length native protein, subject to those modifications that do not change its specific properties. In particular, the term “protein” encompasses protein isoforms, i.e., variants that are encoded by the same gene, but that differ in their pI or MW, or both. Such isoforms can differ in their amino acid sequence (e.g., as a result of alternative slicing or limited proteolysis), or in the alternative, may arise from differential post-translational modification (e.g., glycosylation, acylation or phosphorylation).

The term “protein analog”, as used herein, refers to a polypeptide that possesses a similar or identical function as a parent polypeptide but has an amino acid sequence that differs in at least some respect from that of the parent. In certain embodiments of the invention, a protein analog shares at least a particular characteristic sequence with the parent polypeptide. In some embodiments, such a characteristic sequence is at least 3, 4, 5, 6, 7, 8, 9, 10, 11, 12, 13, 14, 15 16, 17, 18, 19, 20, 21, 22, 23, 24, 25 or more amino acids long. In some embodiments, a characteristic sequence comprises required sequence elements, which may be one or more amino acids long, separated by regions of variability. In some embodiments, a characteristic sequence includes positions in which a particular amino acid residue is required; in some embodiments, a characteristic sequence includes positions in which more than one different amino acid is allowed, but not any amino acid is allowed. In some embodiments, a protein analog shares at least 30%, 35%, 40%, 45%, 50%, 55%, 60%, 65%, 70%, 75%, 80%, 85%, 90%, 91%, 92%, 93%, 94%, 95%, 96%, 97%, 98%, 99%, or more overall sequence identity with the parent polypeptide. Thus, any polypeptide that retains activity and shares at least about 30-40% overall sequence identity, often greater than about 50%, 60%, 70%, or 80%, and further usually including at least one region of much higher identity, often greater than 90%, 96%, 97%, 98% or 99% in one or more highly conserved regions usually encompassing at least 3-4 and often up to 20 or more amino acids, with the parent polypeptide, is encompassed in the term “protein analog”.

The term “protein fragment”, as used herein, refers to a polypeptide whose amino acid sequence is identical to a portion of that of a parent polypeptide. Typically, a protein fragment has an amino acid sequence comprising a stretch of at least 5 amino acid residues found in the parent polypeptide. A protein fragment may or may not possess a functional activity of the full-length parent polypeptide.

The term “biologically active”, refers to an agent that has a designated biological activity. In some embodiments, the term is applied to protein variants, analogs, or fragments in order to designate those that share a biological activity (e.g., ability to specifically bind to cancer cells and/or to be internalized into cancer cells) of the parent polypeptide.

The term “homologous” (or “homology”), as used herein, refers to a degree of identity between two polypeptides, or between two nucleic acid molecules. As is known in the art, when a position in both compared sequences is occupied by the same base or amino acid monomer subunit, then the respective molecules are said to be homologous at that position. The percentage of homology between two sequences corresponds to the number of matching or homologous positions shared by the two sequences divided by the number of positions compared and multiplied by 100. Generally, a comparison is made when two sequences are aligned to give maximum homology. Homologous amino acid sequences share identical or similar amino acid residues. Similar residues are conservative substitutions for, or “allowed point mutations” of, corresponding amino acid residues in a reference sequence. “Conservative substitutions” of a residue in a reference sequence are substitutions that are physically or functionally similar to the corresponding reference residue, e.g., that have a similar size, shape, electric charge, chemical properties, including the ability to form covalent or hydrogen bonds, or the like. Particularly preferred conservative substitutions are those fulfilling the criteria defined for an “accepted point mutation” by Dayhoff et al. (“Atlas of Protein Sequence and Structure”, 1978, Nat. Biomed. Res. Foundation, Washington, D.C., Suppl. 3, 22: 354-352).

The term “fusion protein” refers to a polypeptide comprising two or more proteins or fragments thereof linked by a covalent bond via their individual peptide backbones. In some embodiments, a fusion protein generated through genetic expression of a polynucleotide molecule encoding those proteins.

The term “small molecule” refers to chemical compounds (e.g., organic compounds) that typically have a molecular weight less than about 5,000 daltons (Da). In many embodiments, small molecules have a molecular weight less than about 2,500 Da, less than about 1,000 Da, or less than about 500 Da. In some embodiments, small molecules are not polymers. In some particular embodiments, small molecules are not peptides. In some embodiments, small molecules are biologically active. Small molecules are produced by biological systems (e.g., cells or organisms), or may be chemically synthesized in a laboratory.

### DETAILED DESCRIPTION OF CERTAIN PREFERRED EMBODIMENTS

As mentioned above, the present invention provides compositions and methods for improving the delivery and/or administration of drugs. In particular, the present invention provides conjugates comprising a toxin moiety (e.g., chlorotoxin) associated with a therapeutic agent; and methods for using these conjugate in the treatment of patients. Advantages of administration of inventive conjugates include, among others, selectivity for target cells (including particularly for cancer cells), cellular internalization and retention.

### Conjugates

A conjugate generally is a compound resulting from association (e.g., binding, interaction, or coupling) of at least two molecules. As already mentioned above, a conjugate of the present invention generally comprises at least one toxin moiety (e.g., a chlorotoxin moiety) associated with a therapeutic agent.

The association between a toxin moiety and a therapeutic agent within a conjugate may be covalent or non-covalent. Irrespective of the nature of the association between the toxin moiety and therapeutic agent, the association is preferably selective, specific and strong enough so that the conjugate does not dissociate before or during transport to and into cells. Association between a toxin moiety and a therapeutic moiety may be achieved using any chemical, biochemical, enzymatic, or genetic coupling known to one skilled in the art.

In certain embodiments, association between the toxin moiety and therapeutic agent is non-covalent. Examples of non-covalent associations include, but are not limited to, hydrophobic interactions, electrostatic interactions, dipole interactions, van der Waals interactions, and hydrogen bonding.

In certain embodiments, association between the toxin moiety and therapeutic agent is covalent. As will be appreciated by those skilled in the art, a therapeutic agent and toxin moiety may be attached to each other either directly or indirectly (e.g., through a linker, as discussed below).

In certain embodiments, a therapeutic agent and a toxin moiety are directly, covalently, linked to each other. Such direct covalent binding can be achieved via amide, ester, carbon-carbon, disulfide, carbamate, ether, thioether, urea, amine, or carbonate bonds. Such covalent binding can be achieved, for example, by taking advantage of functional groups present on the therapeutic agent and/or the toxin moiety. Suitable functional groups that can be used to attach two moieties together include, but are not limited to, amines, anhydrides, hydroxyl groups, carboxyl groups, thiols, and the like. In certain embodiments, a functional group of one moiety is activated for coupling to the other moiety. For example, an activating agent, such as a carbodiimide, can be used to effect such a coupling. A wide variety of activating agents are known in the art and are suitable for forming a provided conjugate.

In other embodiments, a therapeutic agent and a toxin moiety are indirectly covalently linked to each other via a linker group. This can be accomplished by using any number of stable bifunctional agents well known in the art, including homofunctional and heterofunctional agents (for examples of such agents, see e.g., Pierce Catalog and Handbook). The use of a bifunctional agent differs from the use of an activating agent in that the former results in a linking moiety being present in the resulting conjugate, whereas the latter results in a direct coupling between two moieties involved in the reaction. The role of the bifunctional agent may be to allow the reaction between the two otherwise inert moieties. Alternatively or additionally, the bifunctional agent, which becomes part of the reaction product may be selected such that it confers some degree of conformational flexibility to the conjugate (e.g., the bifunctional agent comprises a straight alkyl chain containing several atoms, for example, the straight alkyl chain contains between 2 and 10 carbon atoms). Alternatively or additionally, the bifunctional agent may be selected such that the linkage formed between the therapeutic agent and toxin moiety is cleavable, e.g. hydrolysable (for examples of such linkers, see e.g. U.S. Pat. Nos. 5,773,001; 5,739,116 and 5,877,296, each of which is incorporated herein by reference in its entirety). Such linkers are for example preferably used when higher activity of the drug is observed after hydrolysis of the toxin moiety. Exemplary mechanisms by which a drug is cleaved from the toxin moiety include hydrolysis in the acidic pH of the lysosomes (hydrazones, acetals, and cis-aconitate-like amides), peptide cleavage by lysosomal enzymes (the cathepsins and other lysosomal enzymes), and reduction of disulfides. Another mechanism by which a drug is cleaved from the toxin bioconjugate includes hydrolysis at physiological pH extra—or intracellularly. This mechanism applies when the crosslinker used to couple the therapeutic agent to the toxin moiety is a biodegradable/bioerodible entity, such as polydextran and the like.

For example, hydrazone-containing conjugates can be made with introduced carbonyl groups that provide the desired drug-release properties. Conjugates can also be made with a linker that comprises an alkyl chain with a disulfide group at one end and a hydrazine derivative at the other end.

Linkers containing functional groups other than hydrazones also have the potential to be cleaved in the acidic milieu of lysosomes. For example, conjugates can be made from thiol-reactive linkers that contain a group other than a hydrazone that is cleavable intracellularly, such as esters, amides, and acetals/ketals. Ketals made from a 5 to 7 member ring ketone that has one of the oxygen atoms attached to the anti-cancer agent and the other to a linker for toxin attachment can also be used.

Another example of class of pH-sensitive linkers are the cis-aconitates, which have a carboxylic acid group juxtaposed to an amide group. The carboxylic acid accelerates amide hydrolysis in the acidic lysosomes. Linkers that achieve a similar type of hydrolysis rate acceleration with several other types of structures can also be used.

Another potential release method for drug-toxin conjugates is the enzymatic hydrolysis of peptides by the lysosomal enzymes. In one example, a peptidic toxin is attached via an amide bond to para-aminobenzyl alcohol and then a carbamate or carbonate is made between the benzyl alcohol and the therapeutic agent. Cleavage of the peptide leads to collapse of the amino benzyl carbamate or carbonate, and release of the therapeutic agent. In another example, a phenol can be cleaved by collapse of the linker instead of the carbamate. In another variation, disulfide reduction is used to initiate the collapse of a para-mercaptobenzyl carbamate or carbonate.

Many therapeutic agents, in particular anti-cancer agents, have little, if any, solubility in water and that can limit drug loading on the conjugate due to aggregation of the therapeutic agent. One approach to overcoming this is to add solubilizing groups to the linker. Conjugates made with a linker consisting of PEG (polyethylene glycol) and a dipeptide can be used, including, for example, those having a PEG di-acid thiol-acid, or maleimide-acid attached to the toxin moiety, a dipeptide spacer, and an amide bound to a therapeutic agent. Another example is conjugates that are made with a PEG-containing linker disulfide bound to an therapeutic agent and an amide bound to the toxin moiety. Approaches that incorporated PEG groups may be beneficial in overcoming aggregation and limits in drug loading.

In embodiments where a therapeutic moiety within a chlorotoxin conjugate is a protein, a polypeptide or a peptide, the chlorotoxin conjugate may be a fusion protein. As already defined above, a fusion protein is a molecule comprising two or more proteins or peptides linked by a covalent bond via their individual peptide backbones. Fusion proteins used in methods of the present invention can be produced by any suitable method known in the art. For example, they can be produced by direct protein synthetic methods using a polypeptide synthesizer. Alternatively, PCR amplification of gene fragments can be carried out using anchor primers which give rise to complementary overhangs between two consecutive gene fragments that can subsequently be annealed and re-amplified to generate a chimeric gene sequence. Fusion proteins can be obtained by standard recombinant methods (see, for example, Maniatis et al. “Molecular Cloning: A Laboratory Manual”, 2^{nd} Ed., 1989, Cold Spring Harbor Laboratory, Cold Spring, N.Y.). These methods generally comprise (1) construction of a nucleic acid molecule that encodes the desired fusion protein; (2) insertion of the nucleic acid molecule into a recombinant expression vector; (3) transformation of a suitable host cell with the expression vector; and (4) expression of the fusion protein in the host cell. Fusion proteins produced by such methods may be recovered and isolated, either directly from the culture medium or by lysis of the cells, as known in the art. Many methods for purifying proteins produced by transformed host cells are well-known in the art. These include, but are not limited to, precipitation, centrifugation, gel filtration, and (ion-exchange, reverse-phase, and affinity) column chromatography. Other purification methods have been described (see, for example, Deutscher et al. “Guide to Protein Purification” in Methods in Enzymology, 1990, Vol. 182, Academic Press).

As can readily be appreciated by those skilled in the art, a conjugate of the present invention can comprise any number of toxin moieties and any number of therapeutic agent molecules, associated to one another by any number of different ways. The design of a conjugate will be influenced by its intended purpose(s) and the properties that are desirable in the particular context of its use. Selection of a method to associate or bind a toxin moiety to a therapeutic agent to form a conjugate is within the knowledge of one skilled in the art and will generally depend on the nature of the association desired between the moieties (i.e., covalent vs. non-covalent and/or cleavable vs. non-cleavable), the nature of the toxin moiety and therapeutic agent, the presence and nature of functional chemical groups on the moieties involved, and the like.

### Toxins

Conjugates of the present invention comprise at least one toxin moiety. As used herein, the term “toxin moiety” refers to a toxin that specifically binds to cells, particularly tumor/cancer cells, and that gets internalized into these cells. A toxin moiety will often exhibit high affinity, selectivity and/or specificity for particular cells, i.e., it specifically and/or efficiently recognizes, interacts with, binds to, or labels the cells under the conditions or circumstances of its exposure to the cells. When part of a conjugate, the toxin moiety confers at least some of its properties to the conjugate, and the conjugate becomes “targeted” to cells (e.g., tumor/cancer cells) and penetrates into the cells. Preferably, toxin moieties are stable entities that retain their selectivity/specificity and internalization properties under in vivo conditions.

In many embodiments of the present invention, toxin moieties are selected from the group consisting of chlorotoxin, a biologically active chlorotoxin subunit, or a chlorotoxin derivative.

In certain embodiments, the term “chlorotoxin moiety” refers to the full-length, 36 amino acid polypeptide naturally derived from Leiurus quinquestritus scorpion venom (DeBin et al., Am. J. Physiol., 1993, 264: C361-369), which comprises the amino acid sequence of native chlorotoxin as set forth the in SEQ ID NO. 1 of International Application No. WO 2003/101474, the contents of which are incorporated herein by reference. The term “chlorotoxin” includes polypeptides comprising SEQ ID NO. 1 which have been synthetically or recombinantly produced, such as those disclosed in U.S. Pat. No. 6,319,891 (which is incorporated herein by reference in its entirety).

A “biologically active chlorotoxin subunit” is a peptide that comprises less than the 36 amino acids of a chlorotoxin and which retains the ability of chlorotoxin to specifically bind to tumor/cancer cells compared to normal cells, and to get internalized into these tumor/cancer cells.

As used herein, the term “chlorotoxin derivative” refers to any of a wide variety of derivatives, analogs, variants, polypeptide fragments and mimetics of chlorotoxin and related peptides which retain the ability of chlorotoxin to specifically bind to tumor/cancer cells compared to normal cells, and to get internalized into these tumor/cancer cells. Examples of chlorotoxin derivatives include, but are not limited to, peptide variants of chlorotoxin, peptide fragments of chlorotoxin, for example, fragments comprising or consisting of contiguous 10-mer peptides of SEQ ID No. 1, 2, 3, 4, 5, 6, or 7 as set forth in International Application No. WO 2003/101474 or comprising residues 10-18 or 21-30 of SEQ ID No. 1 as set forth in International Application No. WO 2003/101474, core binding sequences, and peptide mimetics.

Examples of chlorotoxin derivatives include peptides having a fragment of the amino acid sequence set forth in SEQ ID No. 1 of International Application No. WO 2003/101474, having at least about 7, 8, 9, 10, 15, 20, 25, 30 or 35 contiguous amino acid residues, associated with the activity of chlorotoxin. Such fragments may contain functional regions of the chlorotoxin peptide, identified as regions of the amino acid sequence which correspond to known peptide domains, as well as regions of pronounced hydrophilicity. Such fragments may also include two core sequences linked to one another, in any order, with intervening amino acid removed or replaced by a linker.

Chlorotoxin derivatives include polypeptides comprising a conservative or non-conservative substitution of at least one amino acid residue when the derivative sequence and the chlorotoxin sequence are maximally aligned. The substitution may be one which enhances at least one property or function of chlorotoxin, inhibits at least one property or function of chlorotoxin, or is neutral to at least one property or function of chlorotoxin. As used herein, a “property or function” of chlorotoxin includes, but is not limited to, the ability to arrest abnormal cell growth, ability to cause paralysis in a subject, ability to specifically bind to a tumor/cancer cell when compared to a normal cell, ability to be internalized into a tumor/cancer cell, and ability to kill a tumor/cancer cell. The tumor/cancer cell may be in vitro, ex vivo, in vitro, a primary isolate from a subject, a cultured cell, or a cell line.

Examples of chlorotoxin derivatives suitable for use in the practice of the present invention are described in International Application No. WO 2003/101474. Particular examples include polypeptides that comprise or consist of SEQ ID NO. 8 or SEQ ID NO. 13 as set forth in this International Application, as well as variants, analogs, and derivatives thereof.

Other examples of chlorotoxin derivatives include those polypeptides containing pre-determined mutations by, e.g., homologous recombination, site-directed or PCR mutagenesis, and the alleles or other naturally-occurring variants of the family of peptides; and derivatives wherein the peptide has been covalently modified by substitution, chemical, enzymatic or other appropriate means with a moiety other than a naturally-occurring amino acid (for example a detectable moiety such as enzyme or a radioisotope).

Chlorotoxin and peptide derivatives thereof can be prepared using any of a wide variety of methods, including standard solid phase (or solution phase) peptide synthesis methods, as is known in the art. In addition, the nucleic acids encoding these peptides may be synthesized using commercially available oligonucleotide synthesis instrumentation and the proteins may be produced recombinantly using standard recombinant production systems.

Other suitable chlorotoxin derivatives include peptide mimetics that mimic the three-dimensional structure of chlorotoxin. Such peptide mimetics may have significant advantages over naturally occurring peptides including, for example, more economical production, greater chemical stability, enhanced pharmacological properties (half-life, absorption, potency, efficacy, etc), altered specificity (e.g., broad-spectrum biological activities, reduced antigenicity and others).

In certain embodiments, mimetics are molecules that mimic elements of chlorotoxin peptide secondary structure. Peptide backbone of proteins exists mainly to orient amino acid side chains in such a way as to facilitate molecular interactions, such as those of antibody and antigen. A peptide mimetic is expected to permit molecular interactions similar to the natural molecule. Peptide analogs are commonly used in the pharmaceutical industry as non-peptide drugs with properties analogous to those of the template peptide. These types of compounds are also referred to as peptide mimetics or peptidomimetics (see, for example, Fauchere, Adv. Drug Res., 1986, 15: 29-69; Veber & Freidinger, Trends Neurosci., 1985, 8: 392-396; Evans et al., J. Med. Chem., 1987, 30: 1229-1239) and are usually developed with the aid of computerized molecular modeling.

Generally, peptide mimetics are structurally similar to a paradigm polypeptide (i.e., a polypeptide that has a biochemical property or pharmacological activity), but have one or more peptide linkages optionally replaced by a non-peptide linkage. The use of peptide mimetics can be enhanced through the use of combinatorial chemistry to create drug libraries. The design of peptide mimetics can be aided by identifying amino acid mutations that increase or decrease the binding of a peptide to, for example, a tumor cell. Approaches that can be used include the yeast two hybrid method (see, for example, Chien et al., Proc. Natl. Acad. Sci. USA, 1991, 88: 9578-9582) and using the phase display method. The two hybrid method detects protein-protein interactions in yeast (Field et al., Nature, 1989, 340: 245-246). The phage display method detects the interaction between an immobilized protein and a protein that is expressed on the surface of phages such as lambda and M13 (Amberg et al., Strategies, 1993, 6: 2-4; Hogrefe et al., Gene, 1993, 128: 119-126). These methods allow positive and negative selection of peptide-protein interactions and the identification of the sequences that determine these interactions.

In certain embodiments, the term “toxin moiety” refers to polypeptide toxins of other scorpion species that display similar or related activity to chlorotoxin described above. As used herein, the term “similar or related activity to chlorotoxin” refers, in particular, to the selectivity/specificity for tumor/cancer cells and the ability to be internalized into a tumor/cancer cell. Examples of suitable related scorpion toxins include, but are not limited to toxins or related peptides of scorpion origin, that display amino acid and/or nucleotide sequence identity to chlorotoxin. Examples of related scorpion toxins include, but are not limited to, CT neurotoxin from Mesobuthus martenssi (GenBank Accession No. AAD473730), Neurotoxin BmK 41-2 from Buthus martensii karsch (GenBank Accession No. A59356), Neurotoxin Bm12-b from Buthus martensii (GenBank Accession No. AAK16444), Probable Toxin LGH 8/6 from Leiurus quinquestriatus hebraeu (GenBank Accession No. P55966), Small toxin from Mesubutus tamulus sindicus (GenBank Accession No. P15229).

Related scorpion toxins suitable for use in the present invention comprise polypeptides that have an amino acid sequence of at least 65%, 75%, 80%, 85%, 90%, 91%, 92%, 93%, 94% 95%, 96%, 97%, 98%, 99% or greater sequence identity with the entire chlorotoxin sequence as set forth in SEQ ID No. 1 of International Application No. WO 2003/101474. In certain embodiments, related scorpion toxins include those scorpion toxins that have a sequence homologous to SEQ ID NO. 8 or SEQ ID NO. 13 of chlorotoxin, as set forth in International Application No. WO 2003/101474.

In certain embodiments, a toxin moiety within an inventive conjugate is labeled. Labeling usually involves non-covalent attachment or covalent attachment (directly or indirectly through a spacer, e.g., a amide group), of one or more labels, preferably to non-interfering positions on the peptide sequence. Such non-interfering positions are positions that do not participate in the specific binding of the toxin moiety to tumor cells and/or to the internalization of the toxin moiety to tumor cells. In preferred embodiments, labeling does not substantially interfere with the desired biological or pharmacological activity of the toxin moiety.

The role of a label or detectable agent is to facilitate detection of the conjugate comprising the toxin moiety. Preferably, the detectable agent is selected such that it generates a signal which can be measured and whose intensity is related to the amount of toxin moiety.

Any of a wide variety of detectable agents can be used in the practice of the present invention. Suitable detectable agents include, but arc not limited to: various ligands, radionuclides; fluorescent dyes; chemiluminescent agents; microparticles; enzymes; colorimetric labels and the like. In certain embodiments, a toxin moiety is labeled with an isotope. For example, a toxin moiety may be isotopically-labeled (i.e., may contain one or more atoms that have been replaced by an atom having an atomic mass or mass number different from the atomic mass or mass number usually found in nature) or an isotope may be attached to the toxin molecule. Examples of isotopes that can be incorporated into toxin moieties include isotopes of hydrogen, carbon, fluorine, phosphorous, iodine, copper, rhenium, indium, yttrium, technetium and lutetium (i.e., ³H, ¹⁴C, ¹⁸F, ¹⁹F, ³²P, ³⁵S, ¹³⁵I, ¹²⁵I, ¹²³I, ⁶⁴Cu, ¹⁸⁷Re, ¹¹¹In, ⁹⁰Y, ^{99m}Tc, ¹⁷⁷Lu). In some embodiments, metal isotopes are non-covalently attached to the toxin moiety by chelation. Examples of chelation include chelation of a metal isotope to a poly-His region fused to a toxin moiety.

In certain embodiment, the toxin moiety is labeled with a metal such as gadolinoum (Gd) either through a covalent bonding or through chelation, as described above.

Such labeled toxin moiety may be useful as radiotracers for positron emission tomography (PET) imaging or for single photon emission computerized tomography (SPECT).

### Therapeutic Agents

In conjugates provided by the present invention, a toxin moiety (e.g., a chlorotoxin moiety) is associated with a therapeutic agent. In certain preferred embodiments, a therapeutic agent is an anti-cancer agent. Suitable anti-cancer agents include any of a large variety of substances, molecules, compounds, agents or factors that are directly or indirectly toxic or detrimental to cancer cells.

As will be recognized by one of ordinary skill in the art, an anti-cancer agent suitable for use in the practice of the present invention may be a synthetic or natural compound; a single molecule or a complex of different molecules. Suitable anti-cancer agents can belong to any of various classes of compounds including, but not limited to, small molecules, peptides, saccharides, steroids, antibodies, fusion proteins, antisense polynucleotides, ribozymes, small interfering RNAs, peptidomimetics, and the like. Similarly, suitable anti-cancer agents can be found among any of a variety of classes of anti-cancer agents including, but not limited to, alkylating agents, anti-metabolite drugs, anti-mitotic antibiotics, alkaloidal anti-tumor agents, hormones and anti-hormones, interferons, non-steroidal anti-inflammatory drugs, and various other anti-tumor agents.

Particularly suitable anti-cancer agents are agents that cause undesirable side effects due to poor selectivity/specificity for cancer cells; agents that undergo no or poor cellular uptake and/or retention; agents that are associated with cellular drug resistance; and agents that cannot be readily formulated for administration to cancer patients due to poor water solubility, aggregation, and the like.

Examples of suitable anti-cancer agents that can be used in conjugates of the present invention are described in more detail below.

### Poorly Water Soluble Anti-Cancer Drugs

In certain embodiments, an anti-cancer agent within an inventive conjugate is a poorly water soluble compound. As will be recognized by one skilled in the art, a wide variety of poorly water soluble anti-cancer agents are suitable for use in the present invention.

For example, an anti-cancer agent may be selected among taxanes, which are recognized as effective agents in the treatment of many solid tumors that are refractory to other anti-neoplastic agents. The two currently approved taxanes are paclitaxel (TAXOL) and docetaxel (TAXOTERE). Paclitaxel, docetaxel, and other taxanes act by enhancing the polymerization of tubulin, an essential protein in the formation of spindle microtubules. This results in the formation of very stable, non-functional tubules, which inhibits cell replication and leads to cell death.

Paclitaxel is very poorly water soluble, and therefore, cannot be practically formulated with water for intravenous administration. Some formulations of TAXOL for injection or intravenous infusion have been developed using Cremophor EL (polyoxyethylated castor oil) as a drug carrier. However, Cremophor EL is itself toxic, and is considered to be, at least in part, responsible for the hypersensitivity reactions (severe skin rashes, hives, flushing, dyspnea, tacchycardia and others) associated with administration of such preparations. To avoid such side effects, pre-medication is often prescribed along with paclitaxel formulations containing Cremophor. Docetaxel, which is an analog of paclitaxel, is like paclitaxel poorly soluble in water. The currently most preferred solvent used to dissolve docetaxel for pharmaceutical use is polysorbate 80 (TWEEN 80). In addition to causing hypersensitivity reactions in patients, TWEEN 80 cannot be used with PVC delivery apparatus, because of its tendency to leach diethylhexyl phthalate, which is highly toxic.

A conjugate according to the present invention comprising a taxane and a toxin (e.g., chlorotoxin) moiety can be used as an improved delivery method to avoids the use of solvents and carriers that induce adverse reactions in patients.

In another example, an anti-cancer agent within an inventive conjugate may belong to the enediyne family of antibiotics. As a family, the enediyne antibiotics are the most potent, anti-tumor agents discovered so far. Some members are 1000 times more potent than adriamycin, one of the most effective, clinically used anti-tumor antibiotics (Y. S. Zhen et al., J. Antibiot., 1989, 42: 1294-1298). For example, an anti-cancer agent within an inventive conjugate may be a member of the enediyne family of calicheamicins. Originally isolated from a broth extract of the soil microorganism Micromonospora echinospora ssp. calichensis, the calicheamicins were detected in a screen for potent DNA damaging agents (M. D. Lee et al., J. Am. Chem. Soc., 1987, 109: 3464-3466; M. D. Lee et al., J. Am. Chem. Soc., 1987, 109: 3466-3468; W. M. Maiese et al., J. Antibiot., 1989, 42: 558-563; M.D. Lee et al., J. Antibiot., 1989, 42: 1070-1087).

Calicheamicins are characterized by a complex, rigid bicyclic enediyne allylic trisulfide core structure linked through glycosyl bonds to an oligosaccharide chain. The oligosaccharide portion contains a number of substituted sugar derivatives, and a substituted tetrahydropyran ring. The enediyne containing core (or aglycone) and carbohydrate portions of calicheamicins have been reported to carry out different roles in the biological activity of these molecules. It is generally believed that the core portion cleaves DNA, whereas the oligosaccharide portion of the calicheamicins serves as a recognition and delivery system and guides the drug to a double-stranded DNA minor groove in which the drug anchors itself (“Enediyne Antibiotics as Antitumor Agents”, Doyle and Borders, 1995, Marcel-Dekker: New York;). Double-stranded DNA cleavage is a type of damage that is usually non-repairable or non-easily repairable for the cell and is most often lethal.

Because of their chemical and biological properties, several analogues of the calicheamicins have been tested in preclinical models as potential anti-tumor agents. Their development as single agent therapies has not been pursued because of delayed toxicities that limit the therapeutic dose range for treatment. However, their potency makes them particularly useful for targeted chemotherapy.

Other examples of suitable poorly water soluble anti-cancer agents include tamoxifen and BCNU. Tamoxifen has been used with varying degrees of success to treat a variety of estrogen receptor positive carcinomas such as breast cancer, endometrial carcinoma, prostate carcinoma, ovarian carcinoma, renal carcinoma, melanoma, colorectal tumors, desmoid tumors, pancreatic carcinoma, and pituitary tumors. In addition to being limited by poor water solubility, chemotherapy using tamoxifen can cause side effects such as cellular drug resistance. BCNU (1,3-bis(2-chloroethyl)-1-nitrosourea) is well known for its anti-tumor properties and, since 1972, it has been charted by the National Cancer Institute for use against brain tumors, colon cancer, Hodgkin's Disease, lung cancer and multiple myeloma. However, the efficient use of this anti-cancer drug is also compromised by its low solubility.

Anti-Cancer Agents Associated with Drug Resistance

In certain embodiments of the present invention, a toxin conjugate comprises an anti-cancer agent associated with drug resistance. As used herein, the term “anti-cancer agent associated with drug resistance” refers to any chemotherapeutics to which cancer cells are or can become resistant. As already mentioned above, resistance to an anti-cancer agent can be due to many factors and can operate by different mechanisms. Administration of a conjugate of the present invention comprising a toxin (e.g., chlorotoxin moiety) and an anti-cancer agent associated with drug resistance can enhance cellular uptake of the anti-cancer agent and carry it into tumor cells, e.g., resistant tumor cells.

Any of a wide variety of anti-cancer agents associated with drug resistance are suitable for use in the present invention. For example, the anti-cancer agent associated with drug resistance may be methotrexate. Methotrexate, a widely used cancer drug, is an analogue of folic acid and blocks important steps in the synthesis of tetrahydrofolic acid which itself is a critical source of compounds utilized in the synthesis of thymidylate, a building block that is specific and therefore especially critical for DNA synthesis. Methotrexate-induced drug resistance is linked to a deficiency in cellular uptake of that drug.

Other examples of suitable anti-cancer agents include purine and pyrimidine analogs that are associated with drug resistance due to inadequate intracellular activation of the drug through loss of enzymatic activity. An example of such a purine analog is 6-mercaptopurine (6-MP). A common cause of tumor cell resistance to 6-MP is the loss of the enzyme hypoxanthine guanine phosphoribosyl transferase (HGPRT) which activates 6-MP into its corresponding nucleotide, 6-mercaptophosphoribosylpurine (6-MPRP), the lethal form of the drug. The resistance could be overcome if 6-MPRP itself could be introduced into the cell. Although this compound is commercially available, it has not yet been used therapeutically in cancer treatment because it is not adequately transported into living cells. Association of 6-MPRP to a toxin moiety according to the present invention would dramatically increase its ability to cross the cell membrane. Thioguanine is another example of anti-cancer agent that is associated with drug resistance due to lack of the enzyme HGPRT.

Examples of pyrimidine analogs that are associated with drug resistance due to inadequate intracellular activation include cytosine arabinoside and adenosine arabinoside which are activated by the enzyme deoxycytidine kinase (DOCK) to the lethal forms cytosine diphosphate and adenosine diphosphate, respectively. A toxin moiety (e.g., chlorotoxin) can be coupled to the activated form of such pyrimidine analogs to enhance their cellular uptake and overcome cellular drug resistance.

Other examples of anti-cancer agents associated with drug resistance include, but are not limited to, 5-fluorouracil, fluorodeoxyuridine, cytosine, arabinoside, vinblastin, vincristin, daunorubicin, doxorubicin, actinomycin, and bleomycin.

### Other Anti-Cancer Agents

In other embodiments, an anti-cancer agent is selected from alkylating drugs (mechlorethamine, chlorambucil, cyclophosphamide, melphalan, ifosfamide), antimetabolites (methotrexate), purine antagonists and pyrimidine antagonists (6-mercaptopurine, 5-fluorouracil, cytarabile, gemcitabine), spindle poisons (vinblastine, vincristine, vinorelbine, paclitaxel), podophyllotoxins (etoposide, irinotecan, topotecan), antibiotics (doxorubicin, bleomycin, mitomycin), nitrosoureas (carmustine, lomustine), inorganic ions (cisplatin, carboplatin), enzymes (asparaginase), and hormones (tamoxifen, leuprolide, flutamide, and megestrol), to name a few. For a more comprehensive discussion of updated cancer therapies see, http://www.cancer.gov/, a list of the FDA approved oncology drugs at http://www.fda.gov/cder/cancer/druglistframe.htm, and The Merck Manual, Seventeenth Ed. 1999, the entire contents of which are hereby incorporated by reference.

### Nucleic Acid Agents

In certain embodiments, a therapeutic (e.g., anti-cancer) agent within an inventive conjugate is a nucleic acid agent.

Numerous cancers and tumors have been shown to be associated with varying degrees of genetic impairment, such as point mutations, gene deletions, or duplications. Many new strategies for the treatment of cancer, such as “antisense”, “antigene”, and “RNA interference” have been developed to modulate the expression of genes (A. Kalota et al., Cancer Biol. Ther., 2004, 3: 4-12; Y. Nakata et al., Crit. Rev. Eukaryot. Gene Expr., 2005, 15: 163-182; V. Wacheck and U. Zangmeister-Wittke, Crit. Rev. Oncol. Hematol., 2006, 59: 65-73; A. Kolata et al., Handb. Exp. Pharmacol., 2006, 173: 173-196). These approaches utilize, for example, antisense nucleic acids, ribozymes, triplex agents, or short interfering RNAs (siRNAs) to block the transcription or translation of a specific mRNA or DNA of a target gene, either by masking that mRNA with an antisense nucleic acid or DNA with a triplex agent, by cleaving the nucleotide sequence with a ribozyme, or by destruction of the mRNA, through a complex mechanism involved in RNA-interference. In all of these strategies, mainly oligonucleotides are used as active agents, although small molecules and other structures have also been applied. While the oligonucleotide-based strategies for modulating gene expression have a huge potential for the treatment of some cancers, pharmacological applications of oligonucleotides have been hindered mainly by the ineffective delivery of these compounds to their sites of action within cancer cells. (P. Herdewijn et al., Antisense Nucleic Acids Drug Dev., 2000, 10: 297-310; Y. Shoji and H. Nakashima, Curr. Charm. Des., 2004, 10: 785-796; A. W Tong et al., Curr. Opin. Mol. Ther., 2005, 7: 114-124).

Conjugates are provided herein that comprise a toxin moiety (e.g., chlorotoxin moiety) and a nucleic acid molecule that is useful as a therapeutic (e.g., anti-cancer) agent. A variety of chemical types and structural forms of nucleic acid can be suitable for such strategies. These include, by way of non-limiting example, DNA, including single-stranded (ssDNA) and double-stranded (dsDNA); RNA, including, but not limited to ssRNA, dsRNA, tRNA, mRNA, rRNA, enzymatic RNA; RNA:DNA hybrids, triplexed DNA (e.g., dsDNA in association with a short oligonucleotide), and the like.

In some embodiments of the present invention, the nucleic acid agent present in an inventive conjugate is between about 5 and 2000 nucleotides long. In some embodiments, the nucleic acid agent is at least about 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40 41, 42, 43, 44, 45, 46, 47, 48, 49, 50 or more nucleotides long. In some embodiments, the nucleic acid agent is less than about 2000, 1900, 1800, 1700, 1600, 1500, 1400, 1300, 1200, 1100, 1000, 900, 800, 700, 600, 500, 450, 400, 350, 300, 250, 200, 150, 100, 50, 45, 40, 35, 30, 25, 20 or fewer nucleotides long.

In some embodiments, a nucleic acid agent present in a conjugate of the present invention comprises a promoter and/or other sequences that regulate transcription. In some embodiments, a nucleic acid agent present in a conjugate of the present invention comprises an origin of replication and/or other sequences that regulate replication. In some embodiments, a nucleic acid agent present in a conjugate of the present invention does not include a promoter and/or an origin of replication.

Nucleic acid anti-cancer agents suitable for use in the practice of the present invention include those agents that target genes associated with tumorigenesis and cell growth or cell transformation (e.g., proto-oncogenes, which code for proteins that stimulate cell division), angiogenic/anti-angiogenic genes, tumor suppressor genes (which code for proteins that suppress cell division), genes encoding proteins associated with tumor growth and/or tumor migration, and suicide genes which induce apoptosis or other forms of cell death, especially suicide genes that are most active in rapidly dividing cells.

Examples of gene sequences associated with tumorigenesis and/or cell transformation include MLL fusion genes, BCR-ABL, TEL-AML1, EWS-FL11, TLS-FUS, PAX3-FKHR, Bcl-2, AML1-ETO, AML1-MTG8, Ras, Fos PDGF, RET, APC, NF-1, Rb, p53, MDM2 and the like; overexpressed sequences such as multidrug resistance genes; cyclins; beta-Catenin; telomerase genes; c-myc, n-myc, Bcl-2, Erb-B1 and Erb-B2; and mutated sequences such as Ras, Mos, Raf, and Met. Examples of tumor suppressor genes include, but are not limited to, p53, p21, RB1, WT1, NF1, VHL, APC, DAP kinase, p16, ARF, Neurofibromin, and PTEN. Examples of genes that can be targeted by nucleic acid molecules useful in anti-cancer therapy include genes encoding proteins associated with tumor migration such as integrins, selectins and metalloproteinases; anti-angiogenic genes encoding proteins that promote the formation of new vessels such as Vascular Endothelial Growth Factor (VEGF) or VEGFr; anti-angiogenic genes encoding proteins that inhibit neovascularization such as endostatin, angiostatin, and VEGF-R2; and genes encoding proteins such as interleukins, interferon, fibroblast growth factor (α-FGF and β-FGF), insulin-like growth factor (e.g., IGF-1 and IGF-2), Platelet-derived growth factor (PDGF), tumor necrosis factor (TNF), Transforming Growth Factor (e.g., TGF-α and TGF-β), Epidermal growth factor (EGF), Keratinocyte Growth Factor (KGF), stem cell factor and its receptor c-Kit (SCF/c-Kit) ligand, CD40L/CD40, VLA-4 VCAM-1, ICAM-1/LFA-1, hyalurin/CD44, and the like. As will be recognized by one skilled in the art, the foregoing examples are not exclusive.

Nucleic acids in conjugates of the present invention may have any of a variety of activities including, for example, as anti-cancer or other therapeutic agents, probes, primers, etc. Nucleic acids in conjugates of the present invention may have enzymatic activity (e.g., ribozyme activity), gene expression inhibitory activity (e.g., as antisense or siRNA agents, etc), and/or other activities. Nucleic acids in conjugates of the present invention may be active themselves or may be vectors that deliver active nucleic acid agents (e.g., through replication and/or transcription of a delivered nucleic acid). For purposes of the present specification, such vector nucleic acids are considered “therapeutic agents” if they encode or otherwise deliver a therapeutically active agent, even if they do not themselves have therapeutic activity.

In certain embodiments, an inventive conjugate comprises a nucleic acid therapeutic agent that comprises or encodes an antisense compound. The terms “antisense compound or agent”, “antisense oligomer”, “antisense oligonucleotide”, and “antisense oligonucleotide analog” are used herein interchangeably, and refer to a sequence of nucleotide bases and a subunit-to-subunit backbone that allows the antisense compound to hybridize to a target sequence in an RNA by Watson-Crick base pairing to form an RNA oligomer heteroduplex within the target sequence. The oligomer may have exact sequence complementarity within the target sequence or near complementarity. Such antisense oligomers may block or inhibit translation of the mRNA containing the target sequence, or inhibit gene transcription. Antisense oligomers may bind to double-stranded or single-stranded sequences.

Examples of antisense oligonucleotides suitable for use in the practice of the present invention include, for example, those mentioned in the following reviews: R. A Stahel et al., Lung Cancer, 2003, 41: S81-S88; K. F. Pirollo et al., Pharmacol. Ther., 2003, 99: 55-77; A. C. Stephens and R. P. Rivers, Curr. Opin. Mol. Ther., 2003, 5: 118-122; N. M. Dean and C. F. Bennett, Oncogene, 2003, 22: 9087-9096; N. Schiavone et al., Curr. Pharm. Des., 2004, 10: 769-784; L. Vidal et al., Eur. J. Cancer, 2005, 41: 2812-2818; T. Aboul-Fadl, Curr. Med. Chem., 2005, 12: 2193-2214; M. E. Gleave and B. P. Monia, Nat. Rev. Cancer, 2005, 5: 468-479; Y. S. Cho-Chung, Curr. Pharm. Des., 2005, 11: 2811-2823; E. Rayburn et al., Lett. Drug Design & Discov., 2005, 2: 1-18; E. R. Rayburn et al., Expert Opin. Emerg. Drugs, 2006, 11: 337-352; I. Tamm and M. Wagner, Mol. Biotechnol., 2006, 33: 221-238 (each of which is incorporated herein by reference in its entirety).

Examples of suitable antisense oligonucleotides include, for example olimerson sodium (also known as Genasense™ or G31239, developed by Genta, Inc., Berkeley Heights, N.J.), a phosphorothioate oligomer targeted towards the initiation codon region of the bel-2 mRNA, which is a potent inhibitor of apoptosis and is overexpressed in many cancer including, follicular lymphomas, breast, colon and prostate cancers, and intermediate/high-grade lymphomas (C. A. Stein et al., Semin. Oncol., 2005, 32: 563-573; S. R. Frankel, Semin. Oncol., 2003, 30: 300-304). Other suitable antisense oligonucleotides include GEM-231 (HYB0165, Hybridon, Inc., Cambridge, Mass.), which is a mixed backbone oligonucleotide directed against cAMP-dependent protein kinase A (PKA) (S. Goel et al., Clin. Cancer Res., 203, 9: 4069-4076); Affinitak (ISIS 3521 or aprinocarsen, ISIS pharmaceuticals, Inc., Carlsbad, Calif.), an antisense inhibitor of PKC-alpha; OGX-011 (Isis 112989, Isis Pharmaceuticals, Inc.), a 2′-methoxyethyl modified antisense oligonucleotide against clusterin, a glycoprotein implicated in the regulation of the cell cycle, tissue remodeling, lipid transport and cell death and which is overexpressed in cancers of breast, prostate and colon; ISIS 5132 (Isis 112989, Isis Pharmaceuticals, Inc.), a phosphorothioate oligonucleotide complementary to a sequence of the 3′-unstranslated region of the c-raf-1 mRNA (S. P. Henry et al., Anticancer Drug Des., 1997, 12: 409-420; B. P. Monia et al., Proc. Natl. Acad. Sci. USA, 1996, 93: 15481-15484; C. M. Rudin et al., Clin. Cancer Res., 2001, 7: 1214-1220); ISIS 2503 (Isis Pharmaceuticals, Inc.), a phosphorothioate oligonucleotide antisense inhibitor of human H-ras mRNA expression (J. Kurreck, Eur. J. Biochem., 2003, 270: 1628-1644); oligonucleotides targeting the X-linked inhibitor of apoptosis protein (XIAP), which blocks a substantial portion of the apoptosis pathway, such as GEM 640 (AEG 35156, Aegera Therapeutics Inc. and Hybridon, Inc.) or targeting survivin, an inhibitor of apoptosis protein (IAP), such as ISIS 23722 (Isis Pharmaceuticals, Inc.), a 2′-O-methoxyethyl chimeric oligonucleotide; MG98, which targets DNA methyl transferase; and GTI-2040 (Lorus Therapeutics, Inc. Toronto, Canada), a 20-mer oligonucleotide that is complementary to a coding region in the mRNA of the R2 small subunit component of human ribonucleotide reductase.

Other suitable antisense oligonucleotides include antisense oligonucleotides that are being developed against Her-2/neu, c-Myb, c-Myc, and c-Raf (see, for example, A. Biroccio et al., Oncogene, 2003, 22: 6579-6588; Y. Lee et al., Cancer Res., 2003, 63: 2802-2811; B. Lu et al., Cancer Res., 2004, 64: 2840-2845; K. F. Pirollo et al., Pharmacol. Ther., 2003, 99: 55-77; and A. Rait et al., Ann. N.Y. Acad. Sci., 2003, 1002: 78-89).

In certain embodiments, an inventive conjugate of the present invention comprises a nucleic acid anti-cancer agent that comprises or encodes an interfering RNA molecule. The terms “interfering RNA” and “interfering RNA molecule” are used herein interchangeably, and refer to an RNA molecule that can inhibit or downregulate gene expression or silence a gene in a sequence-specific manner, for example by mediating RNA interference (RNAi). RNA interference (RNAi) is an evolutionarily conserved, sequence-specific mechanism triggered by double-stranded RNA (dsRNA) that induces degradation of complementary target single-stranded mRNA and “silencing” of the corresponding translated sequences (McManus and Sharp, 2002, Nature Rev. Genet., 2002, 3: 737). RNAi functions by enzymatic cleavage of longer dsRNA strands into biologically active “short-interfering RNA” (siRNA) sequences of about 21-23 nucleotides in length (Elbashir et al., Genes Dev., 2001, 15: 188). RNA interference has emerged as a promising approach for therapy of cancer.

An interfering RNA suitable for use in the practice of the present invention can be provided in any of several forms. For example, an interfering RNA can be provided as one or more of an isolated short interfering RNA (siRNA), double-stranded RNA (dsRNA), micro-RNA (miRNA), or short hairpin RNA (shRNA).

Examples of interfering RNA molecules suitable for use in the present invention include, for example, the iRNAs cited in the following reviews: O. Milhavet et al., Pharmacol. Rev., 2003, 55: 629-648; F. Bi et al., Curr. Gene. Ther., 2003, 3: 411-417; P. Y. Lu et al., Curr. Opin. Mol. Ther., 2003, 5: 225-234; I. Friedrich et al., Semin. Cancer Biol., 2004, 14: 223-230; M. Izquierdo, Cancer Gene Ther., 2005, 12: 217-227; P. Y. Lu et al., Adv. Genet., 2005, 54: 117-142; G. R. Devi, Cancer Gene Ther., 2006, 13: 819-829; M. A. Behlke, Mol. Ther., 2006, 13: 644-670; and L. N. Putral et al., Drug News Perspect., 2006, 19: 317-324 (each of which is incorporated herein by reference in its entirety).

Other examples of suitable interfering RNA molecules include, but are not limited to, p53 interfering RNAs (e.g., T. R. Brummelkamp et al., Science, 2002, 296: 550-553; M. T. Hemman et al., Nat. Genet., 2003, 33: 396-400); interfering RNAs that target the bcr-abl fusion, which is associated with development of chronic myeloid leukemia and acute lymphoblastic leukemia (e.g., M. Scherr et al., Blood, 2003, 101: 1566-1569; M. J. Li et al., Oligonucleotides, 2003, 13: 401-409), interfering RNAs that inhibit expression of NPM-ALK, a protein that is found in 75% of anaplastic large cell lymphomas and leads to expression of a constitutively active kinase associated with tumor formation (U. Ritter et al., Oligonucleotides, 2003, 13: 365-373); interfering RNAs that target oncogenes, such as Raf-1 (T. F. Lou et al., Oligonucleotides, 2003, 13: 313-324), K-Ras (T. R. Brummelkamp et al., Cancer Cell, 2002, 2: 243-247), erbB-2 (G. Yang et al., J. Biol. Chem., 2004, 279: 4339-4345); interfering RNAs that target b-catenin protein, whose over-expression leads to transactivation of the T-cell factor target genes, which is thought to be the main transforming event in colorectal cancer (M. van de Wetering et al., EMBO Rep., 2003, 4: 609-615).

In certain embodiments, an inventive conjugate comprises a nucleic acid therapeutic agent that is a ribozyme. As used herein, the term “ribozyme” refers to a catalytic RNA molecule that can cleave other RNA molecules in a target-specific manner. Ribozymes can be used to downregulate the expression of any undesirable products of genes of interest. Examples of ribozymes that can be used in the practice of the present invention include, but are not limited to, Angiozyme™ (RPI.4610, Sima Therapeutics, Boulder, Colo.), a ribozyme targeting the conserved region of human, mouse, and rat vascular endothelial growth factor receptor (VGEFR)-1 mRNA, and Herzyme (Sima Therapeutics).

### Photosensitizers

In certain embodiments, a therapeutic (e.g., anti-cancer) agent within an inventive conjugate is a photosensitizer used in photodynamic therapy (PDT). In PDT, local or systemic administration of a photosensitizer to a patient is followed by irradiation with light that is absorbed by the photosensitizer in the tissue or organ to be treated. Light absorption by the photosensitizer generates reactive species (e.g., radicals) that are detrimental to cells. For maximal efficacy, a photosensitizer not only has to be in a form suitable for administration, but also in a form that can readily undergo cellular internalization at the target site, preferably with some degree of selectivity over normal tissues.

While some photosensitizer (e.g., Photofrin®, QLT, Inc., Vancouver, BC, Canada) have been delivered successfully as part of a simple aqueous solution, such aqueous solutions may not be suitable for hydrophobic photosensitizer drugs, such as those that have a tetra- or poly-pyrrole-based structure. These drugs have an inherent tendency to aggregate by molecular stacking, which results in a significant reduction in the efficacy of the photosensitization processes (Siggel et al., J. Phys. Chem., 1996, 100: 2070-2075). Approaches to minimize aggregation include liposomal formulations (e.g., for benzoporphyrin derivative monoacid A, BPDMA, Verteporfin®, QLT, Inc., Vancouver, Canada; and zinc phthalocyanine, CIBA-Geigy, Ltd., Basel, Switzerland), and conjugation of photosensitizers to biocompatible block copolymers (Peterson et al., Cancer Res., 1996, 56: 3980-3985) and/or antibodies (Omelyanenko et al., Int. J. Cancer, 1998, 75: 600-608).

Conjugates comprising a toxin moiety associated with a photosensitizer can be used as new delivery systems in PDT. In addition to reducing photosensitizer aggregation, delivery of photosensitizers according to the present invention exhibit other advantages such as increased specificity for target tissues/organ and cellular internalization of the photosensitizer.

Photosensitizers suitable for use in the present invention include any of a variety of synthetic and naturally occurring molecules that have photosensitizing properties useful in PDT. In certain embodiments, the absorption spectrum of the photosensitizer is in the visible range, typically between 350 nm and 1200 nm, preferably between 400 nm and 900 nm, e.g., between 600 nm and 900 nm. Suitable photosensitizers that can be coupled to toxins according to the present invention include, but are not limited to, porphyrins and porphyrin derivatives (e.g., chlorins, bacteriochlorins, isobacteriochlorins, phthalocyanines, and naphthalocyanines); metalloporphyrins, metal lophthalocyanines, angelicins, chalcogenapyrrillium dyes, chlorophylls, coumarins, flavins and related compounds such as alloxazine and riboflavin, fullerenes, pheophorbides, pyropheophorbides, cyanines (e.g., merocyanine 540), pheophytins, sapphyrins, texaphyrins, purpurins, porphycenes, phenothiaziniums, methylene blue derivatives, naphthalimides, nile blue derivatives, quinones, perylenequinones (e.g., hypericins, hypocrellins, and cercosporins), psoralens, quinones, retinoids, rhodamines, thiophenes, verdins, xanthene dyes (e.g., eosins, erythrosins, rose bengals), dimeric and oligomeric forms of porphyrins, and prodrugs such as 5-aminolevulinic acid (R. W. Redmond and J. N. Gamlin, Photochem. Photobiol., 1999, 70: 391-475).

Exemplary photosensitizers suitable for use in the present invention are described in U.S. Pat. Nos. 5,171,741; 5,171,749; 5,173,504; 5,308,608; 5,405,957; 5,512,675; 5,726,304; 5,831,088; 5,929,105; and 5,880,145 (each of which is incorporated herein by reference in its entirety).

### Radiosensitizers

In certain embodiments, a therapeutic (e.g., anti-cancer) agent within an inventive conjugate is a radiosensitizer. As used herein, the term “radiosensitizer” refers to a molecule, compound or agent that makes tumor cells more sensitive to radiation therapy. Administration of a radiosensitizer to a patient receiving radiation therapy generally results in enhancement of the effects of radiation therapy. Ideally, a radiosensitizer exerts its function only on target cells. For ease of use, a radiosensitizer should also be able to find target cells even if it is administered systemically. However, currently available radiosensitizers are typically not selective for tumors, and they are distributed by diffusion in a mammalian body. Toxin conjugates of the present invention can be used as a new delivery system for radiosensitizers.

Radiosensitizers are known in the art. Examples of radiosensitizers suitable for use in the present invention include, but are not limited to, paclitaxel (Taxol®), carboplatin, cisplatin, and oxaliplatin (Amorino et al, Radiat. Oncol. Investig. 1999; 7: 343-352; Choy, Oncology, 1999, 13: 22-38; Safran et al., Cancer Invest., 2001, 19: 1-7; Dionet et al., Anticancer Res., 2002, 22: 721-725; Cividalli et al., Radiat. Oncol. Biol. Phys., 2002, 52: 1092-1098); gemcitabine (Gemzar®) (Choy, Oncology, 2000, 14: 7-14; Mornex and Girard, Annals of Oncology, 2006, 17: 1743-1747); etanidazole (Nitrolmidazole®) (Inanami et al., Int. J. Radiat. Biol., 2002, 78: 267-274); misonidazole (Tamulevicius et al., Br. J. Radiology, 1981, 54: 318-324; Palcic et al., Radiat. Res., 1984, 100: 340-347), tirapazamine (Masunaga et al., Br. J. Radiol., 2006, 79: 991-998; Rischin et al., J. Clin. Oncol., 2001, 19: 535-542; Shulman et al., Int. J. Radiat. Oncol. Biol. Phys., 1999, 44: 349-353); and nucleic acid base derivatives, e.g., halogenated purines or pyrimidines, such as 5-fluorodeoxyuridine (Buchholz et al., Int. J. Radiat. Oncol. Biol. Phys., 1995, 32: 1053-1058).

### Radioisotopes

In certain embodiments, a therapeutic (e.g., anti-cancer) agent within an inventive conjugate is a radioisotope. Examples of suitable radioisotopes include any α-, β- or γ-emitter which, when localized at a tumor site, results in cell destruction (S. E. Order, “Analysis, Results, and Future Prospective of the Therapeutic Use of Radiolabeled Antibody in Cancer Therapy”, Monoclonal Antibodies for Cancer Detection and Therapy, R. W. Baldwin et al. (Eds.), Academic Press, 1985). Examples of such radioisotopes include, but are not limited to, iodine-131 (¹³¹I), iodine-125 (¹²⁵I), bismuth-212 (²¹²Bi), bismuth-213 (²¹³Bi), astatine-211 (²¹¹At), rhenium-186 (¹⁸⁶Re), rhenium-186 (¹⁸⁸Re), phosphorus-32 (³²P), yttrium-90 (⁹⁰Y), samarium-153 (¹⁵³Sm), and lutetium-177 (¹¹⁷Lu).

### Superantigens

In certain embodiments, a therapeutic (e.g., anti-cancer) agent within an inventive conjugate is a superantigen or biologically active portion thereof. Superantigens constitute a group of bacterial and viral proteins that are extremely efficient in activating a large fraction of the T-cell population. Superantigens bind directly to the major histocompatibility complex (MHC) without being processed. In fact, superantigens bind unprocessed outside the antigen-binding groove on the MHC class II molecules, thereby avoiding most of the polymorphism in the conventional peptide-binding site.

A superantigen-based tumor therapeutic approach has been developed for the treatment of solid tumors. In this approach, a targeting moiety, for example, an antibody or antibody fragment, is conjugated to a superantigen, providing a targeted superantigen. If the antibody, or antibody fragment, recognizes a tumor-associated antigen, the targeted superantigen, bound to tumors cells, can trigger superantigen-activated cytotoxic T-cells to kill the tumor cells directly by superantigen-dependent cell mediated cytotoxicity (Søgaard et al., Immunotechnology, 1996, 2: 151-162.

Superantigen-based tumor therapeutics have had some success. For example, fusion proteins with wild-type staphylococcal enterotoxin A (SEA) have been investigated in clinical trials of colorectal and pancreatic cancer (Giantonio et al., J. Clin. Oncol., 1997, 15: 1994-2007; Alpaugh et al., Clin. Cancer Res., 1998, 4: 1903-1914; Cheng et al., J. Clin. Oncol., 2004, 22: 602-609); staphylococcal superantigens of the enterotoxin gene cluster (egc) have been studied for the treatment of non-small cell lung cancer (Terman et al., Clin. Chest Med., 2006, 27: 321-324), and staphylococcal enterotoxin B has been evaluated for the intravesical immunotherapy of superficial bladder cancer (Perabo et al., Int. J. Cancer, 2005, 115: 591-598).

A superantigen, or a biologically active portion thereof, can be associated to a toxin moiety to form a conjugate according to the present invention and used in a therapy, e.g., an anti-cancer therapy, as described herein.

Examples of superantigens suitable for use in the present invention include, but are not limited to staphylococcal enterotoxin (SE) (e.g., staphylococcal enterotoxin A (SEA) or staphylococcal enterotoxin E (SEE)), Streptococcus pyogenes exotoxin (SPE), Staphylococcus aureus toxic shock-syndrome toxin (TSST-1), streptococcal mitogenic exotoxin (SME), streptococcal superantigen (SSA), and staphylococcal superantigens of the enterotoxin gene cluster. As known to one skilled in the art, the three-dimensional structures of the above listed superantigens can be obtained from the Protein Data Bank. Similarly, the nucleic acid sequences and the amino acid sequences of the above listed superantigens and other superantigens can be obtained from GenBank. As will be recognized by one skilled in the art,

### Prodrug Activating Enzymes

In certain embodiments, a conjugate of the present invention may be used in directed enzyme prodrug therapy. In a directed enzyme prodrug therapy approach, a directed/targeted enzyme and a prodrug are administered to a subject, wherein the targeted enzyme is specifically localized to a portion of the subject's body where it converts the prodrug into an active drug. The prodrug can be converted to an active drug in one step (by the targeted enzyme) or in more than one step. For example, the prodrug can be converted to a precursor of an active drug by the targeted enzyme. The precursor can then be converted into the active drug by, for example, the catalytic activity of one or more additional targeted enzymes, one or more non-targeted enzymes administered to the subject, one or more enzymes naturally present in the subject or at the target site in the subject (e.g., a protease, phosphatase, kinase or polymerase), by an agent that is administered to the subject, and/or by a chemical process that is not enzymatically catalyzed (e.g., oxidation, hydrolysis, isomerization, epimerization, etc.).

Different approaches have been used to direct/target the enzyme to the site of interest. For example, in ADEPT (antibody-directed enzyme prodrug therapy), an antibody designed/developed against a tumor antigen is linked to an enzyme and injected in a subject, resulting in selective binding of the enzyme to the tumor. When the discrimination between tumor and normal tissue enzyme levels is sufficient, a prodrug is administered to the subject. The prodrug is converted to its active form by the enzyme, only within the tumor. Selectivity is achieved by the tumor specificity of the antibody and by delaying prodrug administration until there is a large differential between tumor and normal tissue enzyme levels. Early clinical trials are promising and indicate that ADEPT may become an effective treatment for all solid cancers for which tumor-associated or tumor-specific antibodies are known. Tumors have also been targeted with the genes encoding for prodrug activating enzymes. This approach has been called virus-directed enzyme prodrug therapy (VDEPT) or more generally GDEPT (gene-directed enzyme prodrug therapy, and has shown good results in laboratory systems. Other versions of directed enzyme prodrug therapy include PDEPT (polymer-directed enzyme prodrug therapy), LEAPT (lectin-directed enzyme-activated prodrug therapy), and CDEPT (clostridial-directed enzyme prodrug therapy). A conjugate according to the present invention, which comprises a prodrug activating enzyme associated with a toxin moiety, can be used in a similar way.

Examples of enzyme/prodrug/active drug combinations suitable for use in the present invention are described, for example, in Bagshawe et al., Current Opinions in Immunology, 1999, 11: 579-583; Wilman, “Prodrugs in Cancer Therapy”, Biochemical Society Transactions, 14: 375-382, 615^{th} Meeting, Belfast, 1986; Stella et al., “Prodrugs: A Chemical Approach To Targeted Drug Delivery”, in “Directed Drug Delivery”, Borchardt et al., (Eds), pp. 247-267 (Humana Press, 1985). Examples of enzyme/prodrug/active anti-cancer drug combinations are described, for example, in Rooseboom et al., Pharmacol. Reviews, 2004, 56: 53-102.

Examples of prodrug activating enzymes include, but are not limited to, nitroreductase, cytochrome P450, purine-nucleoside phosphorylase, thymidine kinase, alkaline phosphatase, β-glucuronidase, carboxypeptidase, penicillin amidase, β-lactamase, cytosine deaminase, and methionine γ-lyase.

Examples of anti-cancer drugs that can be formed in vivo by activation of a prodrug by a prodrug activating enzyme include, but are not limited to, 5-(aziridin-1-yl)-4-hydroxyl-amino-2-nitro-benzamide, isophosphoramide mustard, phosphoramide mustard, 2-fluoroadenine, 6-methylpurine, ganciclovir-triphosphate nucleotide, etoposide, mitomycin C, p-[N,N-bis(2-chloroethyl)amino]phenol (POM), doxorubicin, oxazolidinone, 9-aminocamptothecin, mustard, methotrexate, benzoic acid mustard, doxorubicin, adriamycin, daunomycin, carminomycin, bleomycins, esperamicins, melphalan, palytoxin, 4-desacetylvinblastine-3-carboxylic acid hydrazide, phenylenediamine mustard, 4′-carboxyphthalato(1,2-cyclohexane-diamine)platinum, taxol, 5-fluorouracil, methylselenol, and carbonothionic difluoride.

### Anti-Angiogenic Agents

In certain embodiments, a therapeutic (e.g., anti-cancer) agent within an inventive conjugate comprises an anti-angiogenic agent. Anti-angiogenic agents suitable for use in the present invention include any molecule, compound or factor that blocks, inhibits, slows down or reduce the process of angiogenesis, or the process by which new blood vessels form by developing from pre-existing vessels. Such a molecule, compound or factor can block angiogenesis by blocking, inhibiting, slowing down or reducing any of the steps involved in angiogenesis, including the steps of (1) dissolution of the membrane of the originating vessel, (2) migration and proliferation of the endothelial cells, and (3) formation of new vascular tube by the migrating cells.

Examples of anti-angiogenic agents include, but arc not limited to, bevacizumab (Avastin®), celecoxib (Celebrex®), endostatin, thalidomide, EMD121974 (Cilengitide), TNP-470, squalamine, combretastatin A4, interferon-α, anti-VEGF antibody, SU5416, SU6668, PTK787/2K 22584, Marimistal, AG3340, COL-3, Neovastat, and BMS-275291.

As will be recognized by one skilled in the art, the specific examples of therapeutic agents cited herein represent only a very small number of the therapeutic agents that are suitable for use in the practice of the present invention.

### Encapsulating Agents

In some embodiments, compositions provided by the present invention include one or more encapsulating agents. In general, an encapsulating agent can be any physiologically tolerable agent that can be used to entrap an entity such as a conjugate or a moiety. By “entrapped” it is meant that the encapsulating agent may encircle or enclose the entity, or an “entrapped” entity may be embedded partially or wholly within the material comprising the encapsulating agent.

In some embodiments, the encapsulating agent is part of the therapeutic moiety, and the toxin moiety is conjugated to the encapsulating agent. In some such embodiments, the toxin moiety is conjugated to the outer surface of the encapsulating agent. In some such embodiments, the toxin moiety is exposed on the environment external to the encapsulating agent. The toxin moiety may be conjugated to the encapsulating agent by a direct interaction (which may be non-covalent or covalent), or it may be conjugated to the encapsulating agent via a linker.

In some embodiments, the conjugate comprising the toxin moiety and the therapeutic moiety is enclosed by the encapsulating agent. The conjugate may be enclosed partially or wholly within a space or environment (for example, an aqueous environment) defined and/or created by the encapsulating agent. In some embodiments, the conjugate is at least partially embedded within the encapsulating agent. For example, if the encapsulating agent comprises lipid membranes, the conjugate may be at least partially embedded within or among lipid molecules in the membrane. In some embodiments, the conjugate is wholly embedded within the encapsulating agent.

A variety of types of encapsulating agents are known in the art, as are methods of using such agents to entrap drugs, biomolecules, and the like. In certain embodiments, the encapsulating agent comprises a small particle having a core and a surface. Such encapsulating agents include, but are not limited to, liposomes, micelles, microparticles, nanoparticles, etc.

Liposomes are typically approximately spherically shaped bilayer structures or vesicles and comprised of natural or synthetic phospholipid membranes. Liposomes may further comprise other membrane components such as cholesterol and protein. The interior core of liposomes typically contain an aqueous solution. Therapeutic agents and/or conjugates may be dissolved in the aqueous solution. As previously mentioned, therapeutic agents and conjugates may be embedded within the membrane of the liposome. Liposomes may be especially useful for delivering agents such as nucleic acid agents (such as those described above), including inhibitory RNAs such as siRNAs.

Micelles are similar to liposomes, except they generally form from a single layer of phospholipids and lack an internal aqueous solution. Reverse micelles that are made to include internal aqueous solution may also be used in accordance with the present invention.

In some embodiments, the particle is a microparticle, at least one dimension of which averages to be smaller than about 1 μm. For example, the smallest dimension of the particles can average about 100 nm, about 120 nm, about 140 nm, about 160 nm, about 180 nm, about 200 nm, about 220 nm, about 240 nm, about 260 nm, about 280 nm, about 300 nm, about 320 nm, about 340 nm, about 360 nm, about 380 nm, about 400 nm, about 420 nm, about 440 nm, about 460 nm, about 480 nm, about 500nm, about 550 nm, about 600 nm, about 650 nm, about 700 nm, about 750 nm, about 800 nm, about 850 nm, about 900 nm, or about 950 nm.

In some embodiments, the particle is a nanoparticle, at least one dimension of which averages to be smaller than about 100 μm. For example, the smallest dimension of the particles can average about 1 nm, about 2 nm, about 3 nm, about 4 nm, about 5 nm, about 6 nm, about 7 nm, about 8 nm, about 9 nm, about 10 nm, about 11 nm, about 12 nm, about 13 nm, about 14 nm, about 15 nm, about 16 nm, about 17 nm, about 18 nm, about 19 nm, about 20 nm, about 22 nm, about 24 nm, about 26 nm, about 28 nm, about 30 nm, about 32 nm, about 34 nm, about 36 nm, about 38 nm, about 40 nm, about 42 nm, about 44 nm, about 46 nm, about 48 nm, about 50 nm, about 55 nm, about 60 nm, about 65 nm, about 70 nm, about 75 nm, about 80 nm, about 85 nm, about 90 nm, about 95 nm, or about 100 nm.

In some embodiments, the core of the particle comprises a material having magnetic resonance activity, which may advantageous in diagnostic and/or therapeutic applications. Materials having magnetic resonance activity include metals and their oxides, such as aluminum-, cobalt-, indium-, iron-, copper-, germanium-, manganese-, nickel-, tin-, titanium-, palladium-, platinum-, selenium-, silicon-, silver-, zinc-, etc containing metals.

In some embodiments, therapeutic agents comprise nucleic acids. Nucleic acids may be enclosed wholly within the encapsulating agent. In some embodiments, nucleic acid agents are embedded within the encapsulating agent. For example, the encapsulating agent may be a liposome and the nucleic agent may be enclosed within the liposome. The nucleic acid agent may be at least partially embedded within the lipid molecules of the liposome.

### II—Pharmaceutical Compositions and Formulations

Conjugates described herein may be administered per se or in the form of a pharmaceutical composition. Accordingly, the present invention provides pharmaceutical compositions comprising an effective amount of at least one inventive conjugate and at least one pharmaceutically acceptable carrier.

A conjugate, or a pharmaceutical composition thereof, may be administered according to the present invention in such amounts and for such a time as is necessary or sufficient to achieve at least one desired result. For example, an inventive pharmaceutical composition can be administered in such amounts and for such a time that it kills cancer cells, reduces tumor size, inhibits tumor growth or metastasis, treats various leukemias, and/or prolongs the survival time of mammals (including humans) with those diseases, or otherwise yields clinical benefit.

Pharmaceutical compositions, according to the present invention, may be administered using any amount and any route of administration effective for achieving the desired therapeutic effect.

The exact amount of pharmaceutical composition to be administered will vary from subject to subject, depending on the species, age, and general condition of the subject, the severity of the condition, and the like (see below).

The optimal pharmaceutical formulation can be varied depending upon the route of administration and desired dosage. Such formulations may influence the physical state, stability, rate of in vivo release, and rate of in vivo clearance of the administered compounds.

Pharmaceutical compositions of the present invention may be formulated in dosage unit form for ease of administration and uniformity of dosage. The expression “unit dosage form”, as used herein, refers to a physically discrete unit of conjugate (with or without one or more additional agents) for the patient to be treated. It will be understood, however, that the total daily usage of compositions of the present invention will be decided by the attending physician within the scope of sound medical judgment.

After formulation with one or more appropriate physiologically acceptable carrier(s) or excipient(s) in a desired dosage, pharmaceutical compositions of the present invention can be administered to humans or other mammals by any suitable route. Various delivery systems are known and can be used to administer such compositions, including, tablets, capsules, injectable solutions, etc. Methods of administration include, but are not limited to, dermal, intradermal, intramuscular, intraperitoneal, intravenous, subcutaneous, intranasal, pulmonary, epidural, ocular, and oral routes. An inventive composition may be administered by any convenient or otherwise appropriate route, for example, by infusion or bolus injection, by absorption through epithelial or mucocutaneous linings (e.g., oral, mucosa, rectal and intestinal mucosa, etc) and may be administered together with other biologically active agents. Administration can be systemic or local.

Injectable preparations, for example, sterile injectable aqueous or oleaginous suspensions may be formulated according to the known art using suitable dispersing or wetting agents, and suspending agents. A sterile injectable preparation may also be a sterile injectable solution, suspension or emulsion in a non-toxic parenterally acceptable diluent or solvent, for example, as a solution in 2,3-butanediol. Among the acceptable vehicles and solvents that may be employed are water, Ringer's solution, U.S.P. and isotonic sodium chloride solution. In addition, sterile, fixed oils are conventionally employed as a solution or suspending medium. For this purpose any bland fixed oil can be employed including synthetic mono- or di-glycerides. Fatty acids such as oleic acid may also be used in the preparation of injectable formulations. Sterile liquid carriers are useful in sterile liquid from compositions for parenteral administration.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. Liquid pharmaceutical compositions which are sterile solutions or suspensions can be administered by, for example, intravenous, intramuscular, intraperitoneal or subcutaneous injection. Injection may be via single push or by gradual infusion (e.g., 30 minute intravenous infusion). Where necessary, the composition may include a local anesthetic to ease pain at the site of injection.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming micro-encapsuled matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations can also be prepared by entrapping the drug in liposomes (also known as lipid vesicles) or microemulsions which are compatible with body tissues.

Liquid dosage forms for oral administration include, but are not limited to, pharmaceutically acceptable emulsions, microemulsions, solutions, suspensions, syrups, elixirs, and pressurized compositions. In addition to the active ingredient (i.e., conjugate), the liquid dosage form may contain inert diluents commonly used in the art such as, for example, water or other solvent, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cotton seed, ground nut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, suspending agents, preservatives, sweetening, flavoring, and perfuming agents, thickening agents, colors, viscosity regulators, stabilizers or osmo-regulators. Suitable examples of liquid carriers for oral administration include water (partially containing additives as above; e.g., cellulose derivatives, such as sodium caboxymethyl cellulose solution), alcohols (including monohydric alcohols and polyhydric alcohols such as glycols) and their derivatives, and oils (e.g., fractionated coconut oil and arachis oil)).

Solid dosage forms for oral administration include, for example, capsules, tablets, pills, powders, and granules. In such solid dosage forms, the active ingredient is mixed with at least one inert, physiologically acceptable excipient or carrier such as sodium citrate or dicalcium phosphate and one or more of: (a) fillers or extenders such as starches, lactose, sucrose, glucose, mannitol, and silicic acid; (b) binders such as, for example, carboxymethylcellulose, alginates, gelatin, polyvinylpyrrolidone, sucrose, and acacia; (c) humectants such as glycerol; (d) disintegrating agents such as agar-agar, calcium carbonate, potato or tapioca starch, alginic acid, certain silicates, and sodium carbonate; (e) solution retarding agents such as paraffin; (f) absorption accelerators such as quaternary ammonium compounds; (g) wetting agents such as, for example, cetyl alcohol and glycerol monostearate; (h) absorbents such as kaolin and bentonite clay; and (i) lubricants such as talc, calcium stearate, magnesium stearate, solid polyethylene glycols, sodium lauryl sulfate, and mixtures thereof. Other excipients suitable for solid formulations include surface modifying agents such as non-ionic and anionic surface modifying agents. Representative examples of surface modifying agents include, but are not limited to, poloxamer 188, benzalkonium chloride, calcium stearate, cetostearyl alcohol, cetomacrogol emulsifying wax, sorbitan esters, colloidal silicon dioxide, phosphates, sodium dodecylsulfate, magnesium aluminum silicate, and triethanolamine. In the case of capsules, tablets and pills, the dosage form may also comprise buffering agents. The amount of solid carrier per solid dosage form will vary widely but preferably will be from about 25 mg to about 1 g.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings, release controlling coatings and other coatings well known in the pharmaceutical formulating art. They may optionally contain opacifying agents and can also be of a composition such that they release the active ingredient(s) only, or preferentially, in a certain part of the intestinal tract, optionally, in a delayed manner. Examples of embedding compositions which can be used include polymeric substances and waxes.

In certain embodiments, it may be desirable to administer an inventive composition locally to an area in need of treatment. This may be achieved, for example, and not by way of limitation, by local infusion during surgery, topically application, by injection, by means of a catheter, by means of suppository, or by means of a skin patch or stent or other implant.

For topical administration, a composition is preferably formulated as a gel, an ointment, a lotion, or a cream which can include carriers such as water, glycerol, alcohol, propylene glycol, fatty alcohols, triglycerides, fatty acid esters, or mineral oil. Other topical carriers include liquid petroleum, isopropyl palmitate, polyethylene glycol, ethanol (95%), polyoxyethylenemonolaurate (5%) in water, or sodium lauryl sulfate (5%) in water. Other materials such as antioxidants, humectants, viscosity stabilizers, and similar agents may be added as necessary. Percutaneous penetration enhancers such as Azone may also be included.

In addition, in certain instances, it is expected that inventive compositions may be disposed within transdermal devices placed upon, in, or under the skin. Such devices include patches, implants, and injections which release the compound onto the skin, by either passive or active release mechanisms. Transdermal administrations include all administrations across the surface of the body and the inner linings of bodily passage including epithelial and mucosal tissues. Such administrations may be carried out using the present compositions in lotions, creams, foams, patches, suspensions, solutions, and suppositories (rectal and vaginal).

Transdermal administration may be accomplished through the use of a transdermal patch containing active ingredient(s) and a carrier that is non-toxic to the skin, and allows the delivery of at least some of the active ingredient(s) for systemic absorption into the bloodstream via the skin. The carrier may take any number of forms such as creams and ointments, pastes, gels, and occlusive devices. Creams and ointments may be viscous liquid or semisolid emulsions of either the oil-in-water or water-in-oil type. Pastes comprised of absorptive powders dispersed in petroleum or hydrophilic petroleum containing active ingredient(s) may also be suitable. A variety of occlusive devices may be used to release active ingredient(s) into the bloodstream such as a semi-permeable membrane covering a reservoir containing the active ingredient(s) with or without a carrier, or a matrix containing the active ingredient.

Suppository formulations may be made from traditional materials, including cocoa butter, with or without the addition of waxes to alter the suppository's melting point, and glycerin. Water soluble suppository bases, such as polyethylene glycols of various molecular weights, may also be used.

Materials and methods for producing various formulations are known in the art and may be adapted for practicing the subject invention.

### III—Dosages and Administration

A treatment according to the present invention may consist of a single dose or a plurality of doses over a period of time.

Administration may be one or multiple times daily, weekly (or at some other multiple day interval) or on an intermittent schedule. For example, an inventive pharmaceutical composition may be administered one or more times per day on a weekly basis for a period of weeks (e.g., 4-10 weeks). Alternatively, an inventive pharmaceutical composition may be administered daily for a period of days (e.g., 1-10 days) following by a period of days (e.g., 1-30 days) without administration, with that cycle repeated a given number of times (e.g., 2-10 cycles).

Administration may be carried out in any convenient manner such as by injection (subcutaneous, intravenous, intramuscular, intraperitoneal, or the like) or oral administration.

Depending on the route of administration, effective doses may be calculated according to the organ function, body weight, or body surface area of the subject to be treated. Optimization of the appropriate dosages can readily be made by one skilled in the art in light of pharmacokinetic data observed in human clinical trials. Final dosage regimen will be determined by the attending physician, considering various factors which modify the action of the drugs, e.g., the drug's specific activity, the severity of the damage and the responsiveness of the patient, the age, condition, body weight, sex and diet of the patient, the severity of any present infection, time of administration, the use (or not) of concomitant therapies, and other clinical factors. As studies are conducted using the inventive combinations, further information will emerge regarding the appropriate dosage levels and duration of treatment.

Typical dosages comprise 1.0 pg/kg body weight to 100 mg/kg body weight. For example, for systemic administration, dosages may be 100.0 ng/kg body weight to 10.0 mg/kg body weight. For direct administration to the site via microinfusion, dosages may be 1 ng/kg body weight to 1 mg/kg body weight.

It will be appreciated that pharmaceutical combinations of the present invention can be employed in combination with additional therapies (i.e., a treatment according to the present invention can be administered concurrently with, prior to, or subsequently to one or more desired therapeutics or medical procedures). The particular combination of therapies (therapeutics or procedures) to employ in such a combination regimen will take into account compatibility of the desired therapeutics and/or procedures and the desired therapeutic effect to be achieved.

For example, methods and compositions of the present invention can be employed together with other procedures including surgery, radiotherapy (e.g., γ-radiation, proton beam radiotherapy, electron beam radiotherapy, proton therapy, brachytherapy, and systemic radioactive isotopes), endocrine therapy, hyperthermia and cryotherapy.

Alternatively or additionally, methods and compositions of the present invention can be employed together with other agents to attenuate any adverse effects (e.g., antiemetics), and/or with other approved chemotherapeutic drugs, including, but not limited to, alkylating drugs (mechlorethamine, chlorambucil, cyclophosphamide, melphalan, ifosfamide), antimetabolites (methotrexate), purine antagonists and pyrimidine antagonists (6-mercaptopurine, 5-fluorouracil, cytarabile, gemcitabine), spindle poisons (vinblastine, vincristine, vinorelbine, paclitaxel), podophyllotoxins (etoposide, irinotecan, topotecan), antibiotics (doxorubicin, bleomycin, mitomycin), nitrosoureas (carmustine, lomustine), inorganic ions (cisplatin, carboplatin), enzymes (asparaginase), and hormones (tamoxifen, leuprolide, flutamide, and megestrol), to name a few. For a more comprehensive discussion of updated cancer therapies see, http://www.cancer.gov/, a list of the FDA approved oncology drugs at http://www.fda.gov/cder/cancer/druglistframe.htm, and The Merck Manual, Seventeenth Ed. 1999, the entire contents of which are hereby incorporated by reference.

Methods and compositions of the present invention can also be employed together with one or more further combinations of cytotoxic agents as part of a treatment regimen, wherein the further combination of cytotoxic agents is selected from: CHOPP (cyclophosphamide, doxorubicin, vincristine, prednisone, and procarbazine); CHOP (cyclophosphamide, doxorubicin, vincristine, and prednisone); COP (cyclophosphamide, vincristine, and prednisone); CAP-BOP (cyclophosphamide, doxorubicin, procarbazine, bleomycin, vincristine, and prednisone); m-BACOD (methotrexate, bleomycin, doxorubicin, cyclophosphamide, vincristine, dexamethasone, and leucovorin); ProMACE-MOPP (prednisone, methotrexate, doxorubicin, cyclophosphamide, etoposide, leucovorin, mechloethamine, vincristine, prednisone, and procarbazine); ProMACE-CytaBOM (prednisone, methotrexate, doxorubicin, cyclophosphamide, etoposide, leucovorin, cytarabine, bleomycin, and vincristine); MACOP-B (methotrexate, doxorubicin, cyclophosphamide, vincristine, prednisone, bleomycin, and leucovorin); MOPP (mechloethamine, vincristine, prednisone, and procarbazine); ABVD (adriamycin/doxorubicin, bleomycin, vinblastine, and dacarbazine); MOPP (mechloethamine, vincristine, prednisone and procarbazine) alternating with ABV (adriamycin/doxorubicin, bleomycin, and vinblastine); MOPP (mechloethamine, vincristine, prednisone, and procarbazine) alternating with ABVD (adriamycin/doxorubicin, bleomycin, vinblastine, and dacarbazine); ChIVPP (chlorambucil, vinblastine, procarbazine, and prednisone); IMVP-16 (ifosfamide, methotrexate, and etoposide); MIME (methyl-gag, ifosfamide, methotrexate, and etoposide); DHAP (dexamethasone, high-dose cytaribine, and cisplatin); ESHAP (etoposide, methylpredisolone, high-dose cytarabine, and cisplatin); CEPP(B) (cyclophosphamide, etoposide, procarbazine, prednisone, and bleomycin); CAMP (lomustine, mitoxantrone, cytarabine, and prednisone); CVP-1 (cyclophosphamide, vincristine, and prednisone), ESHOP (etoposide, methylpredisolone, high-dose cytarabine, vincristine and cisplatin); EPOCH (etoposide, vincristine, and doxorubicin for 96 hours with bolus doses of cyclophosphamide and oral prednisone), ICE (ifosfamide, cyclophosphamide, and etoposide), CEPP(B) (cyclophosphamide, etoposide, procarbazine, prednisone, and bleomycin), CHOP-B (cyclophosphamide, doxorubicin, vincristine, prednisone, and bleomycin), CEPP-B (cyclophosphamide, etoposide, procarbazine, and bleomycin), and P/DOCE (epirubicin or doxorubicin, vincristine, cyclophosphamide, and prednisone).

### IV—Indications

Compositions and methods of the present invention can be used to treat primary and/or metastatic cancers, and other cancerous conditions. For example, compositions and methods of the present invention should be useful for reducing size of solid tumors, inhibiting tumor growth or metastasis, treating various lymphatic cancers, and/or prolonging the survival time of mammals (including humans) suffering from these diseases.

Examples of cancers and cancer conditions that can be treated according to the present invention include, but are not limited to, tumors of the brain and central nervous system (e.g., tumors of the meninges, brain, spinal cord, cranial nerves and other parts of the CNS, such as glioblastomas or medulloblastomas); head and/or neck cancer, breast tumors, tumors of the circulatory system (e.g., heart, mediastinum and pleura, and other intrathoracic organs, vascular tumors, and tumor-associated vascular tissue); tumors of the blood and lymphatic system (e.g., Hodgkin's disease, Non-Hodgkin's disease lymphoma, Burkitt's lymphoma, AIDS-related lymphomas, malignant immunoproliferative diseases, multiple myeloma, and malignant plasma cell neoplasms, lymphoid leukemia, myeloid leukemia, acute or chronic lymphocytic leukemia, monocytic leukemia, other leukemias of specific cell type, leukemia of unspecified cell type, unspecified malignant neoplasms of lymphoid, haematopoietic and related tissues, such as diffuse large cell lymphoma, T-cell lymphoma or cutaneous T-cell lymphoma); tumors of the excretory system (e.g., kidney, renal pelvis, ureter, bladder, and other urinary organs); tumors of the gastrointestinal tract (e.g., esophagus, stomach, small intestine, colon, colorectal, rectosigmoid junction, rectum, anus, and anal canal); tumors involving the liver and intrahepatic bile ducts, gall bladder, and other parts of the biliary tract, pancreas, and other digestive organs; tumors of the oral cavity (e.g., lip, tongue, gum, floor of mouth, palate, parotid gland, salivary glands, tonsil, oropharynx, nasopharynx, puriform sinus, hypopharynx, and other sites of the oral cavity); tumors of the reproductive system (e.g., vulva, vagina, Cervix uteri, uterus, ovary, and other sites associated with female genital organs, placenta, penis, prostate, testis, and other sites associated with male genital organs); tumors of the respiratory tract (e.g., nasal cavity, middle ear, accessory sinuses, larynx, trachea, bronchus and lung, such as small cell lung cancer and non-small cell lung cancer); tumors of the skeletal system (e.g., bone and articular cartilage of limbs, bone articular cartilage and other sites); tumors of the skin (e.g., malignant malonoma of the skin, non-melanoma skin cancer, basal cell carcinoma of skin, squamous cell carcinoma of skin, mesothelioma, Kaposi's sarcoma); and tumors involving other tissues including peripheral nerves and autonomic nervous system, connective and soft tissue, retroperitoneoum and peritoneum, eye and adnexa, thyroid, adrenal gland, and other endocrine glands and related structures, secondary and unspecified malignant neoplasms of lymph nodes, secondary malignant neoplasm of respiratory and digestive systems and secondary malignant neoplasms of other sites.

More specifically, in certain embodiments of the present invention, compositions and methods are used in the treatment of sarcomas. In some embodiments, compositions and methods of the present invention are used in the treatment of bladder cancer, breast cancer, chronic lymphoma leukemia, head and neck cancer, endometrial cancer, Non-Hodgkin's lymphoma, non-small cell lung cancer, ovarian cancer, pancreatic cancer, and prostate cancer.

Tumors that can be treated using compositions and methods of the present invention may be refractory to treatment with other chemotherapeutics. The term “refractory”, when used herein in reference to a tumor means that the tumor (and/or metastases thereof), upon treatment with at least one chemotherapeutic other than an inventive composition, shows no or only weak anti-proliferative response (i.e., no or only weak inhibition of tumor growth) after the treatment of such a chemotherapeutic agent—that is, a tumor that cannot be treated at all or only with unsatisfying results with other (preferably standard) chemotherapeutics. The present invention, where treatment of refractory tumors and the like is mentioned, is to be understood to encompass not only (i) tumors where one or more chemotherapeutics have already failed during treatment of a patient, but also (ii) tumors that can be shown to be refractory by other means, e.g., biopsy and culture in the presence of chemotherapeutics.

### V—Pharmaceutical Packs or Kits

In another aspect, the present invention provides a pharmaceutical pack or kit comprising one or more containers (e.g., vials, ampoules, test tubes, flasks or bottles) containing one or more ingredients of an inventive pharmaceutical composition, allowing administration of a conjugate of the present invention.

Different ingredients of a pharmaceutical pack or kit may be supplied in a solid (e.g., lyophilized) or liquid form. Each ingredient will generally be suitable as aliquoted in its respective container or provided in a concentrated form. Pharmaceutical packs or kits may include media for the reconstitution of lyophilized ingredients. Individual containers of the kit will preferably be maintained in close confinement for commercial sale.

In certain embodiments, a pharmaceutical pack or kit includes one or more additional approved therapeutic agent(s) (e.g., one or more other anti-cancer agents, as described above). Optionally associated with such container(s) can be a notice or package insert in the form prescribed by a governmental agency regulating the manufacture, use or sale of pharmaceutical or biological products, which notice reflects approval by the agency of manufacture, use or sale for human administration. The notice or package insert may contain instructions for use of a pharmaceutical composition according to methods disclosed herein.

An identifier, e.g., a bar code, radio frequency, ID tags, etc., may be present in or on the kit. The identifier can be used for example, to uniquely identify the kit for purposes of quality control, inventory control, tracking movement between workstations, etc.

### EXAMPLES

The following examples describe some of the preferred modes of making and practicing the present invention. However, it should be understood that these examples are for illustrative purposes only and are not meant to limit the scope of the invention. Furthermore, unless the description in an Example is presented in the past tense, the text, like the rest of the specification, is not intended to suggest that experiments were actually performed or data were actually obtained.

### Example 1

### Rapid Uptake and Long-Term Intracellular Localization of TM-601 within Tumor Cells

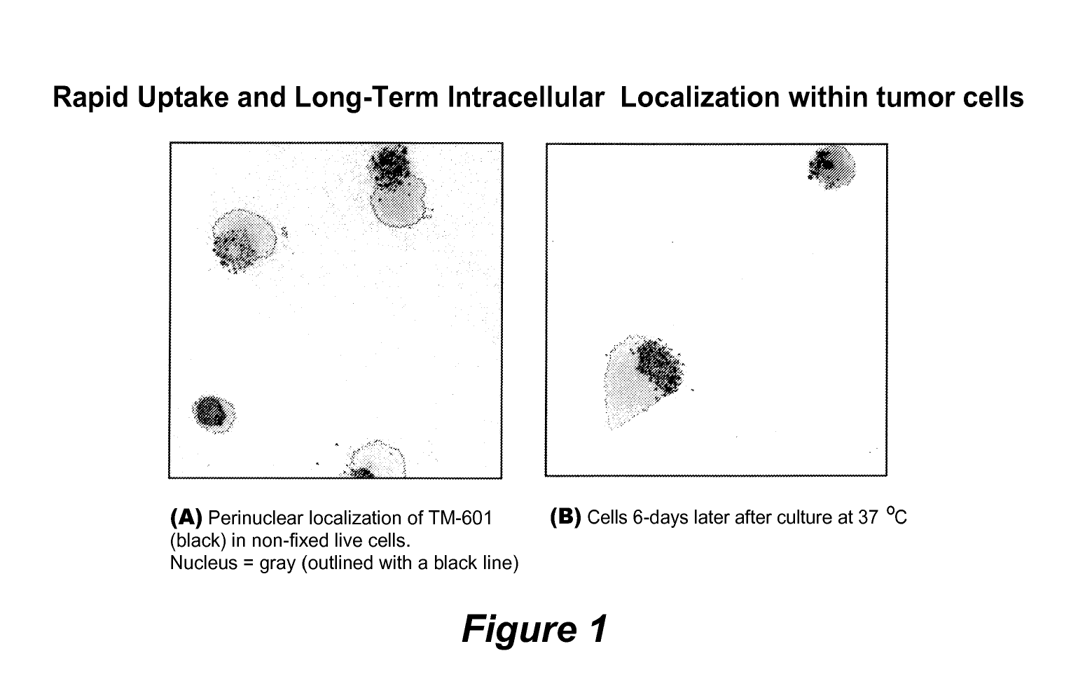

The present example demonstrates the uptake of TM-601 into cancer cells and its stability after uptake. A human glioblastoma cell line, U373, was cultured and stained without fixation for TM-601 uptake by adding to the culture media a fluorescently-tagged TM-601 molecule (labeled in green in FIG. 1). After 24 hours, the media was removed and the cells washed repeatedly to remove residual fluorescently tagged TM-601. For reference, the nucleus was stained with 4′,6-diamidino-2-phenylindole, dihydrochloride (DAPI) (blue) and the photograph in FIG. 1A was taken with a confocal microscope. The cells were then placed in media and cultured at 37° C. for an additional 6 days and the second photograph (FIG. 1B) was taken. The results show that the fluorescently tagged TM-601 that entered the cells during the 24 hour treatment, remained within viable cells for up to 6 days.

### Other Embodiments

Other embodiments of the invention will be apparent to those skilled in the art from a consideration of the specification or practice of the invention disclosed herein. It is intended that the specification and examples be considered as exemplary only, with the true scope of the invention being indicated by the following claims.

What is claimed is:

1. A conjugate comprising at least one toxin moiety associated with at least one therapeutic moiety, wherein the toxin moiety comprises a chlorotoxin, a biologically active fragment thereof or a derivative thereof.

2-11. (canceled)

12. A pharmaceutical composition comprising an effective amount of at least one conjugate of claim 1, or a physiologically tolerable salt thereof, and at least one pharmaceutical acceptable carrier.

13-20. (canceled)

21. A method of treating a cancer patient, the method comprising a step of administering to the patient an effective amount of a conjugate of claim 1.

22. The method of claim 21, wherein administration of the conjugate results in one or more of: higher specific targeting of cancer cells than administration of the therapeutic moiety under substantially identical conditions, higher uptake by cancer cells than administration of the therapeutic moiety under substantially identical conditions, higher retention by cancer cells that administration of the therapeutic moiety under substantially identical conditions, less or less severe undesirable side effects than administration of the therapeutic moiety under substantially identical conditions; and weaker cellular degradation than administration of the therapeutic moiety under substantially identical conditions.

23-32. (canceled)

33. A composition comprising a toxin moiety covalently linked to a nucleic acid agent that is between about 5 and 2000 nucleotides long.

34-49. (canceled)

50. A pharmaceutical composition comprising:
a conjugate of claim 1; and
an encapsulating agent, wherein
the conjugate is entrapped within the encapsulating agent.

51-59. (canceled)

60. A conjugate comprising at least one toxin moiety associated with at least one therapeutic moiety, wherein the toxin moiety comprises a chlorotoxin, biologically active fragment thereof, or derivative thereof having at least 90% sequence identity to SEQ ID NO:1.

61. The conjugate of claim 60, wherein the chlorotoxin, biologically active fragment thereof, or derivative thereof comprises at least seven contiguous amino acid residues associated with the activity of chlorotoxin.

62. The conjugate of claim 60, wherein the toxin moiety comprises at least eight contiguous amino acid residues associated with the activity of chlorotoxin.

63. The conjugate of claim 60, wherein the toxin moiety and therapeutic moiety are covalently associated.

64. The conjugate of claim 63, wherein the toxin moiety and therapeutic moiety are directly covalently associated.

65. The conjugate of claim 63, wherein the toxin moiety and therapeutic moiety are covalently associated through a linker.

66. The conjugate of claim 60, wherein the therapeutic moiety comprises an anti-cancer agent.

67. The conjugate of claim 66, wherein the anti-cancer agent is a member of the group consisting of anti-cancer agents that exhibits poor selectivity/specificity for cancer cells; anti-cancer agents that exhibit poor uptake by cancer cells; anti-cancer agents that exhibit poor retention in cancer cells; anti-cancer agents that exhibit poor water solubility; anti-cancer agents that undergo premature inactivation in cancer cells; anti-cancer agents that undergo impaired activation in cancer cells; anti-cancer agents that undergo extensive cellular degradation; and anti-cancer agents associated with drug resistance.

68. The conjugate of claim 67, wherein the anti-cancer agent exhibits poor water solubility.

69. The conjugate of claim 68, wherein the anti-cancer agent is a taxane.

70. The conjugate of claim 69, wherein the taxane is selected from the group consisting of paclitaxel, docetaxel, and a combination thereof.

71. The conjugate of claim 70, wherein the taxane is paclitaxel.

72. The conjugate of claim 66, wherein the therapeutic moiety is a member of the group consisting of radioisotopes, enzymes, prodrug activating enzymes, radiosensitizers, interfering RNAs, superantigens, anti-angiogenic agents, alkylating agents, purine antagonists, pyrimidine antagonists, plant alkaloids, intercalating antibiotics, aromatase inhibitors, anti-metabolites, mitotic inhibitors, growth factor inhibitors, cell cycle inhibitors, enzymes, topoisomerase inhibitors, biological response modifiers, anti-hormones and anti-androgens.

73. The conjugate of claim 60, wherein the toxin moiety is associated with a label.
